(19) United States
(12) Patent Application Publication
(10) Pub. No.: US 2025/0290091 A1
(43) Pub. Date: Sep. 18, 2025

(54) CONFERRING CYTOPLASMIC MALE STERILITY

(71) Applicant: Syngenta Crop Protection AG, Basel (CH)

(72) Inventors: Esteban Bortiri, Reserach Triangle Park, NC (US); Timothy Kelliher, Research Trianlge Park, NC (US)

(73) Assignee: Syngenta Crop Protection AG, Research Triangle Park, NC (US)

(21) Appl. No.: 18/862,192

(22) PCT Filed: May 15, 2023
(86) PCT No.: PCT/US23/66983

§ 371 Date: Nov. 1, 2024

Related U.S. Application Data
(60) Provisional application No. 63/343,657, filed on May 19, 2022.

(51) Int. Cl. C12N 15/82; C12N 9/22; C12N 15/11

(52) U.S. Cl. CPC C12N 15/8289; C12N 9/226; C12N 15/111; C12N 15/8213; C12N 2310/20

(57) ABSTRACT

Provided herein are methods for conferring cytoplasmic male sterility (CMS) on a plant line. The methods include obtaining a first plant comprising a CMS cytoplasm that is also a haploid inducer (CHIP) and crossing it with a second plant that comprises a desired nuclear genome (DIP). The CHIP also comprises a cenh3 mutation and may contain an anthocyanin marker and a restorer factor. The method further comprises generating progeny from said cross. The progeny produced from the cross of the method is haploid and comprises the CMS cytoplasm of the CHIP as well as the desired nuclear genome of the DIP. The progeny further lacks any anthocyanin marker or restorer factor.

### FIELD OF THE INVENTION

The presently disclosed subject matter relates generally to the field of plant breeding. More specifically, the subject matter relates to methods of conferring CMS (Cytoplasmic Male Sterility) to plant lines. Further, the presently disclosed subject matter relates to specific characteristics in the CMS inducer line (e.g., CENH3 edit) used in the conversion methodology. Additionally, the subject matter generally relates to haploid induction.

### SEQUENCE LISTING

This application is accompanied by a sequence listing entitled 82657-WO-REG-ORG-P-1.xml, created May 5, 2023, which is approximately 187 kilobytes in size. This sequence listing is incorporated herein by reference in its entirety. This sequence listing is submitted herewith via EFS-Web, and is in compliance with 37 C.F.R. § 1.824 (a) (2)-(6) and (b).

### BACKGROUND

Male sterile corn inbreds can be utilized to eliminate the need for detasseling in the production of corn hybrids resulting in significant cost savings. Referred to as Cytoplasmic Male Sterility (CMS), this is a maternally inherited trait that is a useful tool for efficient production of hybrid seed. Three major types of CMS have been identified (CMS-T, CMS-S and CMS-C). Nuclear genes called restorers of fertility (Rf) can over-ride the male-sterile effect of the cytoplasm. Lines with Rf genes produce functional pollen even though they still carry the CMS cytoplasm. Restorer gene loci Rf1 and Rf2 are known to suppress CMS phenotype of CMS-T lines, while the Rf3 gene restores fertility in CMS-S lines. See Laughnan et al. Annu. Rev. Genet. 1983.17:27-48. A restorer locus for CMS-C, Rf4, was mapped to chromosome 8 reported by Sisco et al. In Crop Science Vol. 31 No. 5, p. 1263-1266, 1991. Kohls et al. reported fine mapping of Rf4 at the Maize Genetics Conference in 2010. Also, a patent application published as WO2012047595, incorporated herein by reference, reports the identification of genes and markers associated with Rf4. Breeders produce hybrid seed using a CMS system by developing female lines that carry CMS cytoplasm but lack restorer genes and by developing male lines that carry the appropriate restorer genes. F1 hybrid seed produced by the female lines carry the CMS cytoplasm but yield fertile plants because of the action of the paternally contributed nuclear restorer genes.

Cytotype C was identified as a desirable CMS system for maize, as it generally has stability across environments and genetic backgrounds.

Breeders cross inbred parent lines, one acting as a male and one as a female, in order to form hybrid seed. The process of developing inbred parent lines which are substantially homozygous usually requires a hybrid cross to be selected and self-pollinated (selfed) for numerous generations to become nearly homozygous. This process is time consuming and expensive. To shorten the time to develop homozygous inbreds in maize, rice, wheat, barley, and other crops, breeders may opt to use a haploid inducer line to induce haploid seed production on a hybrid parent. The chromosomes of the haploid plants are then doubled, for example by a chromosome doubling agent such as colchicine, to form doubled haploid homozygous lines.Haploid induction has been observed in numerous plant species, such as sorghum, barley, wheat, and other grasses.

In maize, haploid induction (HI) has been linked to genes MATRILINEAL, ig1, CENH3, and DMP. See generally WO2017/087682, incorporated herein by reference; T. Kelliher, et al., MATRILINEAL, a sperm-specific phospholipase, triggers maize haploid induction, NATURE 542, 105-109 (2017); U.S. Pat. No. 7,439,416, incorporated herein by reference; M Pollacsek, Management of the ig gene for haploid induction in maize, AGRONOMIE, 12:247-251 (1991); U.S. Pat. No. 8,618,354, incorporated herein by reference; see also A.B. Britt and S. Kuppu, Cenh3: An Emerging Player in Haploid Induction Technology, FRONT. PLANT SCI. 7:357 (2016) doi: 10.3389/fpls.2016.00357; and CN111763687 B, incorporated herein by reference. Haploid inducer lines can be created in various ways, for example, through genome editing. As disclosed herein, haploid inducer lines can be generated through a CENH3 mutation, for example. CENH3 is the centromere-specific variant of HISTONE3 (H3) and is required for kinetochore nucleation and spindle attachment in mitosis and meiosis.

In the presently disclosed subject matter, a CMS-C type line with a cenh3 mutation serves as the haploid inducer in a CMS conversion method.

### SUMMARY

A method to convert a maize line with a normal cytotype to a cytoplasmic male sterile (CMS) plant is advantageous for bringing cost and time savings to hybrid seed production. The current industry standard uses trait introgression to cross elite lines in the female heterotic pools into CMS cytoplasm. Disclosed herein is a method to accomplish this conversion to CMS cytoplasm without introgression and instead through a haploid induction process. This process results in 100% nuclear genome conversion into the CMS cytoplasm in a single cross. In this method, a first plant (herein referred to as a “CHIP”), is CMS, heterozygous for a knock-out mutation in CENH3 (i.e., one allele of CENH3 is wildtype; “CENH3+/−”) in its nuclear genome and is a Paternal Haploid Inducer (i.e., it can make paternal haploids). Optionally, the CHIP may also comprise a Restorer Factor (e.g., Rf3, Rf4, Rf10, Rf11, etc.) in its nuclear genome, and if so, is self-fertile despite having a CMS cytotype. Alternatively, the CHIP may also comprise a non-restorer allele (e.g., rf3, rf4, rf10, rf11, rf12). If so, the CHIP is male sterile and requires a maintainer line to continue propagation. The maintainer line is not CMS (i.e., has Normal cytoplasm). Additionally, an optional anthocyanin marker is homozygous in the CHIP.

A second plant, herein referred to as a “DIP”, is not a haploid inducer (i.e., homozygous wildtype for CENH3 (“CENH3+/+”), possesses a normal cytotype (i.e., not cytoplasmic male sterile), and comprises a Desired Nuclear Genome to be converted into a CMS cytoplasm. The DIP is the pollen donor.

The CHIP is crossed with the DIP pollen and offspring include diploid progeny (“F1s”), haploid progeny (comprising only the nuclear genome of the DIP), and aneuploid progeny. Only the haploid progeny is desired and will comprise (i) the haploid nuclear genome (i.e., the desired genome) of the DIP with one wildtype CENH3 allele (“CENH3+/null”), and (ii) the CMS-C cytotype, (iii) further lacking a restorer factor, and optionally (iv) the visual marker.

When used, a visual marker, such as an anthocyanin marker (e.g., R1-nj or R1-scm2), may be employed to distinguish the undesired diploid F1 progeny from the desired haploid progeny. If used, the marker is present in the haploid inducer genome (i.e., the CHIP) in the homozygous state. Aneuploids are distinguished from true haploid progeny by using the genetic markers for the inducer parent, i.e., any putative haploids showing female (inducer) parent markers (indicative of CHIP nuclear DNA) will be discarded as aneuploids. Alternatively, the haploid progeny may be distinguished from the diploids and aneuploids by sequencing, selectable markers, stature, or measurements of ploidy level.

### BRIEF DESCRIPTION OF THE SEQUENCES IN THE SEQUENCE LISTING

SEQ ID NO: 1 the nucleotide sequence of gRNA140 carried by LbCas12a RNP, targeting the second exon of gene ID GRMZM2G158526, in the biolistic bombardment of SYN-INBC34 inbred immature embryos in example 1 and of SYN-INBC34 x SYN-INBC34RS inbred immature embryos in example 2.

SEQ ID NO: 2 is a nucleotide sequence of a primer used in TaqMan assay 3895.

SEQ ID NO: 3 is a nucleotide sequence of a primer used in TaqMan assay 3895

SEQ ID NO: 4 is the nucleotide sequence of the probe used in TaqMan assay 3895.

SEQ ID NO: 5 is the nucleotide sequence representing a 19 base pair deletion in cenh3.

SEQ ID NO: 6 is the nucleotide sequence representing a 10 base pair deletion in cenh3.

SEQ ID NO: 7 is the partial nucleotide sequence of wild type cenh3 (GRMZM2G158526).

SEQ ID NO: 8 is the partial nucleotide sequence of the cenh3 10 base pair deletion mutant (509A115A).

SEQ ID NO: 9 is the partial nucleotide sequence of the cenh3 19 base pair deletion mutant (509A151A).

SEQ ID NO: 10 is the partial amino acid sequence of the cenh3 allele (GRMZM2G158526).

SEQ ID NO: 11 is the partial amino acid sequence of the deduced amino acid sequence for the KD (Kelly Dawe at the University of Georgia) cenh3 allele.

SEQ ID NO: 12 is the partial amino acid sequence of the cenh3 allele with the 10 base pair deletion.

SEQ ID NO: 13 is the partial amino acid sequence of the cenh3 allele with the 19 base pair deletion splice variant a.

SEQ ID NO: 14 is the partial amino acid sequence of the cenh3 allele with the 19 base pair deletion splice variant c.

SEQ ID NO: 15 is the partial amino acid sequence of the cenh3 allele with the 19 base pair deletion splice variant b.

SEQ ID NO: 16 is the partial amino acid sequence of the cenh3 allele with the 19 base pair deletion splice variant d.

SEQ ID NO: 17 is the partial amino acid sequence of the cenh3 allele with the 19 base pair deletion splice variant e.

SEQ ID NO: 18 is the partial amino acid sequence of the cenh3 allele with the 10 base pair deletion splice variant a.

SEQ ID NO: 19 is the partial amino acid sequence of the cenh3 allele with the 10 base pair deletion splice variant b.

SEQ ID NO: 20 is the partial amino acid sequence of the cenh3 allele with the 10 base pair deletion splice variant c.

SEQ ID NO: 21 is the partial amino acid sequence of the cenh3 allele with the 10 base pair deletion splice variant d.

SEQ ID NO: 22 is the partial amino acid sequence of the cenh3 allele with the 10 base pair deletion splice variant e.

SEQ ID NO: 23 is the nucleotide sequence of the forward primer for assay SM0253EQ.

SEQ ID NO: 24 is the nucleotide sequence of the reverse primer for assay SM0253EQ.

SEQ ID NO: 25 is the nucleotide sequence of the probe for assay SM0253EQ.

SEQ ID NO: 26 is the nucleotide sequence of the probe for assay SM0253EQ.

SEQ ID NO: 27 is the nucleotide sequence of the target for assay SM0253EQ.

SEQ ID NO: 28 is the nucleotide sequence of the forward primer for assay SM0093B.

SEQ ID NO: 29 is the nucleotide sequence of the reverse primer for assay SM0093B.

SEQ ID NO: 30 is the nucleotide sequence of the probe for assay SM0093B.

SEQ ID NO: 31 is the nucleotide sequence of the probe for assay SM0093B.

SEQ ID NO: 32 is the nucleotide sequence of the target for assay SM0093B.

SEQ ID NO: 33 is the nucleotide sequence of the forward primer for assay SM0435A.

SEQ ID NO: 34 is the nucleotide sequence of the reverse primer for assay SM0435A.

SEQ ID NO: 35 is the nucleotide sequence of the probe for assay SM0435A.

SEQ ID NO: 36 is the nucleotide sequence of the probe for assay SM0435A.

SEQ ID NO: 37 is the nucleotide sequence of the target for assay SM0435A.

SEQ ID NO: 38 is the nucleotide sequence of the forward primer for assay SM1071CQ.

SEQ ID NO: 39 is the nucleotide sequence of the reverse primer for assay SM1071CQ.

SEQ ID NO: 40 is the nucleotide sequence of the probe for assay SM1071CQ.

SEQ ID NO: 41 is the nucleotide sequence of the probe for assay SM1071CQ.

SEQ ID NO: 42 is the nucleotide sequence of the target for assay SM1071CQ.

SEQ ID NO: 43 is the nucleotide sequence of the forward primer for assay SM1280CQ.

SEQ ID NO: 44 is the nucleotide sequence of the reverse primer for assay SM1280CQ.

SEQ ID NO: 45 is the nucleotide sequence of the probe for assay SM1280CQ.

SEQ ID NO: 46 is the nucleotide sequence of the probe for assay SM1280CQ.

SEQ ID NO: 47 is the nucleotide sequence of the target for assay SM1280CQ.

SEQ ID NO: 48 is the nucleotide sequence of the forward primer for assay SM1447AQ.

SEQ ID NO: 49 is the nucleotide sequence of the reverse primer for assay SM1447AQ.

SEQ ID NO: 50 is the nucleotide sequence of the probe for assay SM1447AQ.

SEQ ID NO: 51 is the nucleotide sequence of the probe for assay SM1447AQ.

SEQ ID NO: 52 is the nucleotide sequence of the target for assay SM1447AQ.

SEQ ID NO: 53 is the nucleotide sequence of the forward primer for assay SM1847AQ.

SEQ ID NO: 54 is the nucleotide sequence of the reverse primer for assay SM1847AQ.

SEQ ID NO: 55 is the nucleotide sequence of the probe for assay SM1847AQ.

SEQ ID NO: 56 is the nucleotide sequence of the probe for assay SM1847AQ.

SEQ ID NO: 57 is the nucleotide sequence of the target for assay SM1847AQ.

SEQ ID NO: 58 is the nucleotide sequence of the forward primer for assay SM1286AQ.

SEQ ID NO: 59 is the nucleotide sequence of the reverse primer for assay SM1286AQ.

SEQ ID NO: 60 is the nucleotide sequence of the probe for assay SM1847AQ.

SEQ ID NO: 61 is the nucleotide sequence of the probe for assay SM1847AQ.

SEQ ID NO: 62 is the nucleotide sequence of the target for assay SM1286AQ.

SEQ ID NO: 63 is the nucleotide sequence of the forward primer for assay SM2513.

SEQ ID NO: 64 is the nucleotide sequence of the reverse primer for assay SM2513.

SEQ ID NO: 65 is the nucleotide sequence of the probe for assay SM2513.

SEQ ID NO: 66 is the nucleotide sequence of the probe for assay SM2513.

SEQ ID NO: 67 is the nucleotide sequence of the target for assay SM2513.

SEQ ID NO: 68 is the nucleotide sequence of the forward primer for assay SM2962.

SEQ ID NO: 69 is the nucleotide sequence of the reverse primer for assay SM2962.

SEQ ID NO: 70 is the nucleotide sequence of the probe for assay SM2962.

SEQ ID NO: 71 is the nucleotide sequence of the probe for assay SM2962.

SEQ ID NO: 72 is the nucleotide sequence of the target for assay SM2962.

SEQ ID NO: 73 is the nucleotide sequence of the forward primer for assay SM2988.

SEQ ID NO: 74 is the nucleotide sequence of the reverse primer for assay SM2988.

SEQ ID NO: 75 is the nucleotide sequence of the probe for assay SM2988.

SEQ ID NO: 76 is the nucleotide sequence of the probe for assay SM2988.

SEQ ID NO: 77 is the nucleotide sequence of the target for assay SM2988.

SEQ ID NO: 78 is the nucleotide sequence of the forward primer for assay SM3400.

SEQ ID NO: 79 is the nucleotide sequence of the reverse primer for assay SM3400.

SEQ ID NO: 80 is the nucleotide sequence of the probe for assay SM3400.

SEQ ID NO: 81 is the nucleotide sequence of the probe for assay SM3400.

SEQ ID NO: 82 is the nucleotide sequence of the target for assay SM3400.

SEQ ID NO: 83 is the nucleotide sequence of the forward primer for assay SM3747.

SEQ ID NO: 84 is the nucleotide sequence of the reverse primer for assay SM3747.

SEQ ID NO: 85 is the nucleotide sequence of the probe for assay SM3747.

SEQ ID NO: 86 is the nucleotide sequence of the probe for assay SM3747.

SEQ ID NO: 87 is the nucleotide sequence of the target for assay SM3747.

SEQ ID NO: 88 is the nucleotide sequence of the forward primer for assay SM4788.

SEQ ID NO: 89 is the nucleotide sequence of the reverse primer for assay SM4788.

SEQ ID NO: 90 is the nucleotide sequence of the probe for assay SM4788.

SEQ ID NO: 91 is the nucleotide sequence of the probe for assay SM4788.

SEQ ID NO: 92 is the nucleotide sequence of the target for assay SM4788.

SEQ ID NO: 93 is the nucleotide sequence of the forward primer for assay SM5515.

SEQ ID NO: 94 is the nucleotide sequence of the reverse primer for assay SM5515.

SEQ ID NO: 95 is the nucleotide sequence of the probe for assay SM5515.

SEQ ID NO: 96 is the nucleotide sequence of the probe for assay SM5515.

SEQ ID NO: 97 is the nucleotide sequence of the target for assay SM5515.

SEQ ID NOs: 98-100 are the nucleotide sequences of the primers and probe for assay PM1901 (wildtype ZmCENH3).

SEQ ID NOs: 101-103 are the nucleotide sequences of the primers and probe for assay PM1909 (mutation in ZmCENH3 comprising a 10 bp deletion).

SEQ ID NOs: 101, 102, and 104 are the nucleotide sequences of the primers and probe for assay PM1913 (mutation in ZmCENH3 comprising a 19 bp deletion).

SEQ ID NOs: 105-108 are the nucleotide sequences of the primers and probes for assay SM0576CQ.

SEQ ID NO: 109-112 are the nucleotide sequences of the primers and probes for assay SM0956IQ.

SEQ ID NO: 113-116 are the nucleotide sequences of the primers and probes for assay SM2669.

SEQ ID NO: 117-120 are the nucleotide sequences of the primers and probes for assay SM2670.

SEQ ID NO: 121-124 are the nucleotide sequences of the primers and probes for assay SM2915.

SEQ ID NO: 125-128 are the nucleotide sequences of the primers and probes for assay SM2916.

SEQ ID NO: 129-132 are the nucleotide sequences of the primers and probes for assay SM6623.

SEQ ID NO: 133-136 are the nucleotide sequences of the primers and probes for assay SM8040.

SEQ ID NO: 137-140 are the nucleotide sequences of the primers and probes for assay SM8091.

SEQ ID NO: 141-144 are the nucleotide sequences of the primers and probes for assay SM2918.

SEQ ID NO: 145-148 are the nucleotide sequences of the primers and probes for assay SM4813.

SEQ ID NO: 149-152 are the nucleotide sequences of the primers and probes for assay SM2914.

SEQ ID NO: 153-156 are the nucleotide sequences of the primers and probes for assay SM4812.

SEQ ID NO: 157-160 are the nucleotide sequences of the primers and probes for assay SM0954BQ.

SEQ ID NO: 161-164 are the nucleotide sequences of the primers and probes for assay SM6568.

SEQ ID NO: 165-168 are the nucleotide sequences of the primers and probes for assay SM0953BQ.

SEQ ID NO: 169-172 are the nucleotide sequences of the primers and probes for assay SM7200.

SEQ ID NO: 173-176 are the nucleotide sequences of the primers and probes for assay SM5665.

SEQ ID NO: 177 is the partial nucleotide sequence of the cenh3 19 base pair deletion mutant from table 1 (509A150A).

### Definitions

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art. While the following terms are believed to be well understood by one of ordinary skill in the art, the following definitions are set forth to facilitate explanation of the presently disclosed subject matter.

Following long-standing patent law convention, the terms “a,” “an,” and “the” refer to “one or more” when used in this application, including the claims. For example, the phrase “a cell” refers to one or more cells, and in some embodiments can refer to a tissue and/or an organ. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to all whole number values between 1 and 100 as well as whole numbers greater than 100.

Unless otherwise indicated, all numbers expressing quantities of ingredients, reaction conditions, and so forth used in the specification and claims are to be understood as being modified in all instances by the term “about.” The term “about,” as used herein when referring to a measurable value such as an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods and/or employ the discloses compositions, nucleic acids, polypeptides, etc. Accordingly, unless indicated to the contrary, the numerical parameters set forth in this specification and attached claims are approximations that can vary depending upon the desired properties sought to be obtained by the presently disclosed subject matter.

As used herein, the term “allele” refers to a variant or an alternative sequence form at a genetic locus. In diploids, a single allele is inherited by a progeny individual separately from each parent at each locus. The two alleles of a given locus present in a diploid organism occupy corresponding places on a pair of homologous chromosomes, although one of ordinary skill in the art understands that the alleles in any particular individual do not necessarily represent all of the alleles that are present in the species.

As used herein, the term “amplified” means the construction of multiple copies of a nucleic acid molecule or multiple copies complementary to the nucleic acid molecule using at least one of the nucleic acid molecules as a template. Amplification systems include the polymerase chain reaction (PCR) system, ligase chain reaction (LCR) system, nucleic acid sequence-based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, PERSING et al., Ed., American Society for Microbiology, Washington, D.C. (1993). The product of amplification is termed an “amplicon.”

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D (e.g., AB, AC, AD, BC, BD, CD, ABC, ABD, and BCD). In some embodiments, one of more of the elements to which the “and/or” refers can also individually be present in single or multiple occurrences in the combinations(s) and/or subcombination(s).

The term “aneuploid” refers to a plant with an abnormal number of chromosomes in a haploid set.

As used herein, the term “backcrossing” or “backcrossed” is understood within the scope of the invention to refer to a process in which a hybrid progeny is repeatedly crossed back to one of the parents.

The terms “bombarding”, “bombardment”, and “biolistic bombardment” refer to the process of accelerating particles towards a target biological sample (e.g., cell, tissue, etc.) to cause wounding of the cell membrane of a cell in the target biological sample and/or entry of the particles into the target biological sample. Methods for biolistic bombardment are known in the art (e.g., U.S. Pat. No. 5,584,807), and are commercially available (e.g., the helium gas-driven accelerator (PDS-1000/He™ from BioRad). The biolistic PDS-1000 Gene Gun (BioRad, Hercules, CA) uses helium pressure to accelerate DNA-coated gold or tungsten microparticles toward target cells.

As used herein, the term “bulk” refers to the process of increasing the number of seeds.

As used herein, the term “cDNA” refers to a single-stranded or a double-stranded DNA that is complementary to and derived from mRNA.

As used herein, the term “CHIP” refers to one of the parents in the original cross of the methodology of the present invention. This parent has a heterozygous cenh3 mutation (C) in its nuclear genome, is a paternal haploid inducer (HI), and as mentioned above, is one of the parents (P). The CHIP is female fertile and CMS male sterile. The CHIP may optionally contain a homozygous restorer factor in its nuclear genome. The CHIP may also optionally contain a homozygous anthocyanin marker in its nuclear genome.

The term “chromosome” is used herein as recognized in the art as meaning the self-replicating genetic structure in the cellular nucleus containing the cellular DNA and bearing the linear array of genes.

The term “comprising,” which is synonymous with “including,” “containing,” and “characterized by,” is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter.

As used herein, the term “cytoswapping” refers to the exchange of cytoplasm from one line to another (e.g., “Normal A” cytoplasm in a maize line swapped into another maize line which was originally “Normal B” cytoplasm).

As used herein, the term “DIP” refers to one of the parents in the original cross of the methodology of the present invention. This parent contains the desired haploid nuclear genome (the “desired parent” or “DIP”). The DIP is self-fertile (homozygous for wild type CENH3) and has a normal cytotype.

As used herein, a plant referred to as “diploid” has two complete sets of chromosomes (2n; one set from each parent).

As used herein, the term “elite line” or “inbred line” refers to any line that has resulted from breeding and selection for superior agronomic performance. An elite line has stable genetics, i.e., it is reasonably or nearly isogenic across its genome. Said another way, an elite line is reasonably or nearly homozygous for all alleles in its genome.

As used herein, the term “expression” when used with reference to a polynucleotide, such as a gene, ORF or portion thereof, or a transgene in plants, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (e.g. if a gene encodes a protein), through “translation” of mRNA. Gene expression can be regulated at many stages in the process. For example, in the case of antisense or dsRNA constructs, respectively, expression may refer to the transcription of the antisense RNA only or the dsRNA only. In embodiments, “expression” refers to the transcription and stable accumulation of sense (mRNA) or functional RNA. “Expression” may also refer to the production of protein.

As used herein, the term “gene” refers to a hereditary unit including a sequence of DNA that occupies a specific location on a chromosome and that contains the genetic instruction for a particular characteristic or trait in an organism. “genome”

As used herein, the term “genotype” refers to the genetic constitution of a cell or organism. An individual's “genotype for a set of genetic markers” includes the specific alleles, for one or more genetic marker loci, present in the individual. As is known in the art, a genotype can relate to a single locus or to multiple loci, whether the loci are related or unrelated and/or are linked or unlinked. In some embodiments, an individual's genotype relates to one or more genes that are related in that the one or more of the genes are involved in the expression of a phenotype of interest (e.g., a quantitative trait as defined herein). Thus, in some embodiments a genotype comprises a sum of one or more alleles present within an individual at one or more genetic loci of a quantitative trait. In some embodiments, a genotype is expressed in terms of a haplotype (defined herein below).

As used herein, the term “germplasm” refers to the totality of the genotypes of a population or another group of individuals (e.g., a species). The term “germplasm” can also refer to plant material; e.g., a group of plants that act as a repository for various alleles. The phrase “adapted germplasm” refers to plant materials of proven genetic superiority; e.g., for a given environment or geo-graphical area, while the phrases “non-adapted germplasm”, “raw germplasm”, and “exotic germplasm” refer to plant materials of unknown or unproven genetic value; e.g., for a given environment or geographical area; as such, the phrase “non-adapted germplasm” refers in some embodiments to plant materials that are not part of an established breeding population and that do not have a known relationship to a member of the established breeding population.

As used herein, a plant referred to as “haploid” has a single set (genome) of chromosomes and the reduced number of chromosomes (In) in the haploid plant is equal to that of the gamete. As used herein, a plant referred to as “doubled haploid” is developed by doubling the haploid set of chromosomes (from In to 2n). A plant or seed that is obtained from a doubled haploid plant that is selfed to any number of generations may still be identified as a doubled haploid plant. A doubled haploid plant is considered a homozygous plant. A plant is considered to be doubled haploid if it is fertile, even if the entire vegetative part of the plant does not consist of the cells with the doubled set of chromosomes; that is, a plant will be considered doubled haploid if it contains viable gametes, even if it is chimeric.

As used herein, “HaploidBC1” refers to the progeny of a haploid plant which has been pollinated with its recurrent parent, so as to effectuate a backcross in the progeny. HaploidBC1 progeny comprise a diploid genome.

As used herein, haploid induction rate (“HIR”) means the number of surviving haploid kernels over the total number of kernels after an ear is pollinated with haploid inducer pollen.

As used herein, the term “heterologous” when used in reference to a gene or nucleic acid refers to a gene encoding a factor that is not in its natural environment (i.e., has been altered by the hand of man). For example, a heterologous gene may include a gene from one species introduced into another species. A heterologous gene may also include a gene native to an organism that has been altered in some way (e.g., mutated, added in multiple copies, linked to a non-native promoter or enhancer polynucleotide, etc.). Heterologous genes further may comprise plant gene polynucleotides that comprise cDNA forms of a plant gene; the cDNAs may be expressed in either a sense (to produce mRNA) or anti-sense orientation (to produce an anti-sense RNA transcript that is complementary to the mRNA transcript). In one aspect of the invention, heterologous genes are distinguished from endogenous plant genes in that the heterologous gene polynucleotide are typically joined to polynucleotides comprising regulatory elements such as promoters that are not found naturally associated with the gene for the protein encoded by the heterologous gene or with plant gene polynucleotide in the chromosome, or are associated with portions of the chromosome not found in nature (e.g., genes expressed in loci where the gene is not normally expressed). Further, in embodiments, a “heterologous” polynucleotide is a polynucleotide not naturally associated with a host cell into which it is introduced, including non-naturally occurring multiple copies of a naturally occurring polynucleotide.

As used herein, the term “heterozygous” means a genetic condition existing when different alleles reside at corresponding loci on homologous chromosomes.

As used herein, the term “homozygous” means a genetic condition existing when identical alleles reside at corresponding loci on homologous chromosomes.

As used herein, the term “human-induced mutation” refers to any mutation that occurs as a result of either direct or indirect human action. This term includes, but is not limited to, mutations obtained by any method of targeted mutagenesis.

As used herein, the term “hybrid” refers to offspring produced by crossing two genetically dissimilar parent plants. The resulting progeny of this cross are a “bi-parental” population. The terms “hybrid”, “hybrid plant”, and “hybrid progeny” in the context of plant breeding refer to a plant that is the offspring of genetically dissimilar parents produced by crossing plants of different lines or breeds or species, including but not limited to the cross between two inbred lines (e.g., a genetically heterozygous or mostly heterozygous individual). The phrase “single cross F1 hybrid” refers to an F1 hybrid produced from a cross between two inbred lines.

The term “identity” or “identical” in the context of two nucleic acid or amino acid sequences, refers to the percentage of identical nucleotides or amino acids in a linear polynucleotide or amino acid sequence of a reference (“query”) sequence (or its complementary strand) as compared to a test (“subject”) sequence when the two sequences are globally aligned. Unless otherwise stated, sequence identity as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/and as described in the following publication: “The EMBL-EBI search and sequence analysis tools APIs in 2019.” Madeira et al. Nucleic Acids Research, June 2019, 47 (W1): W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle. In some embodiments, substantially identical nucleic acid or amino acid sequences may perform substantially the same function.

The phrase “inbred line” refers to a genetically homozygous or nearly homozygous population. An inbred line, for example, can be derived through several cycles of brother/sister breedings or of selfing. In some embodiments, inbred lines breed true for one or more phenotypic traits of interest. An “inbred”, “inbred individual”, or “inbred progeny” is an individual sampled from an inbred line. The term “inbred” means a substantially homozygous individual or line.

As used herein, “introduced” means delivered, expressed, applied, transported, transferred, permeated, or other like term to indicate the delivery, whether of nucleic acid or protein or combination thereof, of a desired object to an object. For example, nucleic acids encoding a site directed nuclease and optionally at least one guide RNA may be introduced into a haploid embryo upon haploid induction. Likewise, extant editing machinery (comprising a site directed nuclease protein and optionally at least one guide RNA) may be introduced to a haploid embryo upon application of appropriate cell-penetrating peptides.

As used herein, the terms “introgression”, “introgressed” and “introgressing” refer to both a natural and artificial process whereby genomic regions of one species, variety or cultivar are moved into the genome of another species, variety or cultivar, by crossing those species. The process may optionally be completed by backcrossing to the recurrent parent.

As used herein, the term “isolated,” when used in the context of the nucleic acid molecules or polynucleotides of the present invention, refers to a polynucleotide that is identified within and isolated/separated from its chromosomal polynucleotide context within the respective source organism. An isolated nucleic acid or polynucleotide is not a nucleic acid as it occurs in its natural context, if it indeed has a naturally occurring counterpart. In contrast, non-isolated nucleic acids are nucleic acids such as DNA and RNA, which are found in the state they exist in nature. For example, a given polynucleotide (e.g., a gene) is found on the host cell chromosome in proximity to neighboring genes. The isolated nucleic acid molecule may be present in single-stranded or double-stranded form. Alternatively, it may contain both the sense and antisense strands (i.e., the nucleic acid molecule may be double-stranded). In a preferred embodiment, the nucleic acid molecules of the present invention are understood to be isolated.

As used herein, the term “knockout mutation” refers to a gene mutation in which expression of said gene is stopped or ‘knocked out’. This mutation can include, but is not limited to, mutations obtained by any method of targeted mutagenesis.

As used herein, the term “locus” refers to a position (e.g., of a gene, a genetic marker, or the like) on a chromosome of a given species.

As used herein, “maternal haploid inducer” refers to a line that produces pollen and, when crossed as a male, results in the gynogenic development of haploid seeds. A “paternal haploid inducer” refers to a line that when used as a female in a cross, results in androgenic development of haploid seeds. A haploid inducer plant can use either of these maternal or paternal mechanisms to derive haploids.

As used herein, the term “maintainer line” refers to a plant line that is male fertile, comprises a normal cytoplasm, is substantially genetically similar (e.g., isogenic) to a CMS plant line, and is used to maintain the stock of the CMS inducer. A maintainer line is preferably homozygous for a non-restorer allele (e.g., rf4) as well as a R1 color marker (e.g., R1-nj or R1-SCM2). Additionally, the maintainer line may comprise a CenH3 mutation. Herein, a maintainer line is used as a male in a cross with a CMS line which may or may not comprise a CenH3 mutation. As used herein, the term “normal cytoplasm” refers to cytotypes that are fertile (as opposed to cytoplasmic male sterile). Maintainer lines may be selfed to increase their seed stock.

As used herein, the term “molecular marker” can be used to refer to a genetic marker, as defined above, or an encoded product thereof (e.g., a protein) used as a point of reference when identifying the presence/absence of a HI-associated locus. A molecular marker can be derived from genomic nucleotide sequences or from expressed nucleotide sequences (e.g., from an RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution (e.g., according to Watson-Crick base pairing rules). This term also refers to the genetic markers that indicate a trait by the absence of the nucleotide sequences complementary to or flanking the marker sequences, such as nucleotide sequences used as probes and/or primers capable of amplifying the marker sequence.

As used herein, the terms “nucleotide sequence,” “polynucleotide,” “nucleic acid sequence,” “nucleic acid molecule,” and “nucleic acid fragment” refer to a polymer of RNA or DNA that is single- or double-stranded, optionally containing synthetic, non-natural, and/or altered nucleotide bases. A “nucleotide” is a monomeric unit from which DNA or RNA polymers are constructed and consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “offspring” refers to any plant resulting as progeny from a vegetative or sexual reproduction from one or more parent plants or descendants thereof. For instance, an offspring plant may be obtained by cloning or selfing of a parent plant or by crossing two parent plants and includes selfings as well as the F1 or F2 or still further generations. An F1 is a first-generation offspring produced from parents at least one of which is used for the first time as donor of a trait, while offsprings of second generation (F2) or subsequent generations (F3, F4, etc.) are specimens produced from selfings of F1's, F2's etc. An F1 may thus be a hybrid resulting from a cross between two true breeding parents (true-breeding is homo-zygous for a trait), while an F2 may be an offspring resulting from self-pollination of said F1 hybrids.

The term “PCR (polymerase chain reaction)” is understood within the scope of the invention to refer to a method of producing relatively large amounts of specific regions of DNA, thereby making possible various analyses that are based on those regions.

As used herein, the term “plant” can refer to a whole plant, any part thereof, or a cell or tissue culture derived from a plant. Thus, the term “plant” can refer to any of: whole plants, plant components or organs (e.g., leaves, stems, roots, etc.), plant tissues, seeds and/or plant cells.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

As used herein, “preserved pollen” refers to pollen collected manually and stored in some manner for future use (See U.S. Application No. 63/289,299, herein incorporated by reference).

As used herein, the term “primer” refers to an oligonucleotide which is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to amplify plant nucleic acids (e.g., using the polymerase chain reaction; PCR).

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

The term “plant cell culture” means cultures of plant units such as, for example, protoplasts, cell culture cells, cells in plant tissues, pollen, pollen tubes, ovules, embryo sacs, zygotes and embryos at various stages of development.

“Plant material” refers to leaves, stems, roots, flowers or flower parts, fruits, pollen, egg cells, zygotes, seeds, cuttings, cell or tissue cultures, or any other part or product of a plant.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any group of plant cells organized into structural and/or functional units. The use of this term in conjunction with, or in the absence of, any specific type of plant tissue as listed above or otherwise embraced by this definition is not intended to be exclusive of any other type of plant tissue.

The term “plant part” indicates a part of a plant, including single cells and cell tissues such as plant cells that are intact in plants, cell clumps and tissue cultures from which plants can be regenerated. Examples of plant parts include, but are not limited to, single cells and tissues from pollen, ovules, leaves, embryos, roots, root tips, anthers, flowers, fruits, stems, shoots, and seeds; as well as pollen, ovules, leaves, embryos, roots, root tips, anthers, flowers, fruits, stems, shoots, scions, rootstocks, seeds, protoplasts, calli, and the like.

As used herein, the terms “phenotype,” “phenotypic trait” or “trait” refer to one or more traits of a plant or plant cell. The phenotype can be observable to the naked eye, or by any other means of evaluation known in the art, e.g., microscopy, biochemical analysis, or an electromechanical assay. In some cases, a phenotype is directly controlled by a single gene or genetic locus (i.e., corresponds to a “single gene trait”). In the case of haploid induction use of color markers, such as R Navajo, and other markers including transgenes visualized by the presences or absences of color within the seed evidence if the seed is an induced haploid seed. The use of R Navajo as a color marker and the use of transgenes is well known in the art as means to detect induction of haploid seed on the female plant. In other cases, a phenotype is the result of interactions among several genes, which in some embodiments also results from an interaction of the plant and/or plant cell with its environment.

As used herein, the term “population” means a genetically heterogeneous collection of plants sharing a common genetic derivation.

As used herein, the term “primer” refers to an oligonucleotide which is capable of annealing to a nucleic acid target (in some embodiments, annealing specifically to a nucleic acid target) allowing a DNA polymerase and/or reverse transcriptase to attach thereto, thereby serving as a point of initiation of DNA synthesis when placed under conditions in which synthesis of a primer extension product is induced (e.g., in the presence of nucleotides and an agent for polymerization such as DNA polymerase and at a suitable temperature and pH). In some embodiments, one or more pluralities of primers are employed to amplify plant nucleic acids (e.g., using the polymerase chain reaction; PCR).

The term “primer”, as used herein, refers to an oligonucleotide which is capable of annealing to the amplification target allowing a DNA polymerase to attach, thereby serving as a point of initiation of DNA synthesis when placed under conditions in which synthesis of primer extension product is induced, e.g., in the presence of nucleotides and an agent for polymerization such as DNA polymerase and at a suitable temperature and pH. The (amplification) primer is preferably single stranded for maximum efficiency in amplification. Preferably, the primer is an oligodeoxyribonucleotide. The primer is generally sufficiently long to prime the synthesis of extension products in the presence of the agent for polymerization. The exact lengths of the primers will depend on many factors, including temperature and composition (A/T and G/C content) of primer. A pair of bi-directional primers consists of one forward and one reverse primer as commonly used in the art of DNA amplification such as in PCR amplification. It will be understood that “primer,” as used herein, may refer to more than one primer, particularly in the case where there is some ambiguity in the information regarding the terminal sequence(s) of the target region to be amplified. Hence, a “primer” includes a collection of primer oligonucleotides containing sequences representing the possible variations in the sequence or includes nucleotides which allow a typical base pairing. The oligonucleotide primers may be prepared by any suitable method. Methods for preparing oligonucleotides of specific sequence are known in the art, and include, for example, cloning and restriction of appropriate sequences, and direct chemical synthesis. Chemical synthesis methods may include, for example, the phospho di- or tri-ester method, the diethylphosphoramidate method and the solid support method disclosed in, for example, U.S. Pat. No. 4,458,066. The primers may be labeled, if desired, by incorporating means detectable by, for instance, spectroscopic, fluorescence, photochemical, biochemical, immunochemical, or chemical means. Template-dependent extension of the oligonucleotide primer(s) is catalyzed by a polymerizing agent in the presence of adequate amounts of the four deoxyribonucleotide triphosphates (dATP, dGTP, dCTP and dTTP, i.e. dNTPs) or analogues, in a reaction medium which is comprised of the appropriate salts, metal cations, and pH buffering system. Suitable polymerizing agents are enzymes known to catalyze primer- and template-dependent DNA synthesis. Known DNA polymerases include, for example, E. coli DNA polymerase I or its Klenow fragment, T4 DNA polymerase, and Taq DNA polymerase. The reaction conditions for catalyzing DNA synthesis with these DNA polymerases are known in the art. The products of the synthesis are duplex molecules consisting of the template strands and the primer extension strands, which include the target sequence. These products, in turn, serve as template for another round of replication. In the second round of replication, the primer extension strand of the first cycle is annealed with its complementary primer; synthesis yields a “short” product which is bound on both the 5′- and the 3′-ends by primer sequences or their complements. Repeated cycles of denaturation, primer annealing, and extension result in the exponential accumulation of the target region defined by the primers. Sufficient cycles are run to achieve the desired amount of polynucleotide containing the target region of nucleic acid. The desired amount may vary and is determined by the function which the product polynucleotide is to serve. The PCR method is well described in handbooks and known to the skilled person. After amplification by PCR, the target polynucleotides may be detected by hybridization with a probe polynucleotide which forms a stable hybrid with that of the target sequence under low, moderate or even highly stringent hybridization and wash conditions. If it is expected that the probes will be essentially completely complementary (i.e., about 99% or greater) to the target sequence, highly stringent conditions may be used. If some mismatching is expected, for example if variant strains are expected with the result that the probe will not be completely complementary, the stringency of hybridization may be lessened. However, conditions are typically chosen which rule out nonspecific/adventitious binding. Conditions, which affect hybridization, and which select against nonspecific binding are known in the art, and are described in, for example, Sambrook and Russell, 2001. Generally, lower salt concentration and higher temperature increase the stringency of hybridization conditions. “PCR primer” is preferably understood within the scope of the present invention to refer to relatively short fragments of single-stranded DNA used in the PCR amplification of specific regions of DNA.

As used herein, the term “probe” refers to a nucleic acid (e.g., a single stranded nucleic acid or a strand of a double stranded or higher order nucleic acid, or a subsequence thereof) that can form a hydrogen-bonded duplex with a complementary sequence in a target nucleic acid sequence. Typically, a probe is of sufficient length to form a stable and sequence-specific duplex molecule with its complement, and as such can be employed in some embodiments to detect a sequence of interest present in a plurality of nucleic acids.

The term “probe” refers to a single-stranded oligonucleotide that will form a hydrogen-bonded duplex with a substantially complementary oligonucleotide in a target nucleic acid analyte or its cDNA derivative.

As used herein, the terms “marker probe” and “probe” refer to a nucleotide sequence or nucleic acid molecule that can be used to detect the presence or absence of a sequence within a larger sequence, e.g., a nucleic acid probe that is complementary to all of or a portion of the marker or marker locus, through nucleic acid hybridization. Marker probes comprising about 8, 10, 15, 20, 30, 40, 50, 60, 70, 80, 90, 100 or more contiguous nucleotides can be used for nucleic acid hybridization.

The term “progeny” refers to the descendant(s) of a particular cross. Typically, progeny result from breeding of two individuals, although some species (particularly some plants and hermaphroditic animals) can be selfed (i.e., the same plant acts as the donor of both male and female gametes). The descendant(s) can be, for example, of the F1, the F2, or any subsequent generation.

As used herein, the terms “progeny” and “progeny plant” refer to a plant generated from vegetative or sexual reproduction from one or more parent plants. In gynogenesis-mediated haploid induction, the haploid embryo on the female parent comprises female chromosomes to the exclusion of male chromosomes—thus it is not a progeny of the male haploid-inducing line. The haploid corn seed typically still has normal triploid endosperm that contains the male genome. The edited haploid progeny and subsequent edited doubled haploid plants and subsequent seed is not the only desired progeny. There is also the seed from the haploid inducer line itself, often carrying the Cas9 transgene, and subsequent plant and seed progeny of the haploid inducing plant. Both the haploid seed and the haploid inducer (self-pollination-derived) seed can be progeny. A progeny plant can be obtained by cloning or selfing a single parent plant, or by crossing two or more parental plants. For instance, a progeny plant can be obtained by cloning or selfing of a parent plant or by crossing two parental plants and include selfings as well as the F1 or F2 or still further generations. An F1 is a first-generation progeny produced from parents at least one of which is used for the first time as donor of a trait, while progeny of second generation (F2) or subsequent generations (F3, F4, and the like) are specimens produced from selfings, intercrosses, backcrosses, and/or other crosses of F1s, F2s, and the like. An F1 can thus be (and in some embodiments is) a hybrid resulting from a cross between two true breeding parents (i.e., parents that are true-breeding are each homozygous for a trait of interest or an allele thereof), while an F2 can be (and in some embodiments is) a progeny resulting from self-pollination of the F1 hybrids.

The terms “R1-nj” and “R1-SCM2” refer to the R1-Navajo and R1-SCM2 anthocyanin markers. These visual markers are useful for distinguishing haploids from diploids (or aneuploids). As described herein, haploids plants are identified as cream-colored while diploids are purple in color.

As used herein, the term “regenerate,” and grammatical variants thereof, refers to the production of a plant from tissue culture.

As used herein, “Restorer factor” or “Fertility restorer” or “Rf” or “restorer allele” refers to a gene or genes in a plant that restores fertility to a male sterile plant. Examples of restorer factor genes include, but are not limited to, Rf3, Rf4, Rf10, Rf11, and Rf12. Plants may be heterozygous or homozygous for one or more restorer factor genes. For example, a plant may contain Rf4 as well as Rf11 but may also be rf10. Another plant in a cross may be rf4 but also be Rf11 and Rf10. Therefore, each plant can contain and lack restorer and non-restorer alleles. Thus, a CMS plant that comprises at least one restorer allele will nevertheless be male fertile.

Non-restorer allele (also referred to as “rf”) means a gene or genes in a plant that do not restore fertility to a male sterile plant. Examples of non-restorer alleles include, but are not limited to, rf3, rf4, rf10, rf11, and rf12. Plants homozygous for non-restorer alleles will be male sterile if the plant possesses CMS.

As used herein, “spontaneous chromosome doubling” (“SCD”) or “spontaneous haploid genome doubling” or “haploid male fertility” or “spontaneous genome doubling” are used interchangeably to describe the doubling of haploid genomes without any intervention. SCD allows for the correct meiotic reduction of chromosomes and subsequent formation of mature pollen. In the present disclosure, SCD was calculated by dividing the number of fertile haploid plants/by the total number of plants.

As used herein, “spontaneously doubled haploid plant” refers to a plant whose florets have undergone spontaneous doubling. Other tissues of a spontaneously doubled haploid plant may retain their haploid state (e.g., root, leaf, stem).

As used herein, the term “targeted mutagenesis” or “mutagenesis strategy” refers to any method of mutagenesis that results in the intentional mutagenesis of a chosen gene. Targeted mutagenesis includes the methods CRISPR, TILLING, TALEN, and other methods not yet discovered but which may be used to achieve the same outcome. As used herein, the term “targeted mutagenesis” or “mutagenesis strategy” refers to any method of mutagenesis that results in the intentional mutagenesis of a chosen gene. Targeted mutagenesis includes the methods CRISPR, TILLING, TALEN, and other methods not yet discovered but which may be used to achieve the same outcome.

As used herein, the term “trait” refers to a phenotype of interest, a gene that contributes to a phenotype of interest, as well as a nucleic acid sequence associated with a gene that contributes to a phenotype of interest. For example, a “HI trait” refers to a haploid induction phenotype as well as a gene (e.g., matl in maize or Os03g27610 in rice) that contributes to a haploid induction and a nucleic acid sequence (e.g., a HI-associated gene product) that is associated with the presence or absence of the haploid induction phenotype.

As used herein, the term “transfected” refers to the introduction of nucleic acids into cells.

As used herein, the term “transgene” refers to a nucleic acid molecule introduced into an organism or one or more of its ancestors by some form of artificial transfer technique. The artificial transfer technique thus creates a “transgenic organism” or a “transgenic cell.” It is understood that the artificial transfer technique can occur in an ancestor organism (or a cell therein and/or that can develop into the ancestor organism) and yet any progeny individual that has the artificially transferred nucleic acid molecule or a fragment thereof is still considered transgenic even if one or more natural and/or assisted breedings result in the artificially transferred nucleic acid molecule being present in the progeny individual.

### DETAILED DESCRIPTION

Described herein is a method for conferring cytoplasmic male sterility (CMS) on a plant line. The method comprises obtaining a first plant comprising a CMS cytoplasm that is a haploid inducer (CHIP), obtaining a second plant comprising a desired nuclear genome (DIP), and crossing the CHIP with the DIP, and generating a progeny from said cross. The resulting progeny comprises the CMS cytoplasm and the desired nuclear genome from the CHIP and DIP, respectively. In an embodiment, the CMS cytoplasm is selected from the group consisting of CMS-C, CMS-S, and CMS-T. In one embodiment, the CMS cytoplasm is CMS-C. In one embodiment, the CHIP is female fertile and CMS male sterile. In another embodiment, the CHIP is female fertile and CMS male fertile. The CHIP is a paternal haploid inducer and comprises a cenh3 mutation. In one embodiment, the cenh3 mutation is a knockout mutation. In one embodiment, the cenh3 knockout mutation is obtained by gene editing. In another embodiment, the cenh3 knockout mutation comprises SEQ ID NO: 5 or SEQ ID NO: 6. In another embodiment, the cenh3 knockout mutation is heterozygous. In yet another embodiment, the cenh3 mutation is edited using CRISPR-Cas12a. In one embodiment, the CRISPR-Cas12a is selected from the group consisting of AsCas12a, LbCas12a, and FnCas12a, MbCas12a, and Mb2Cas12a. In one embodiment, the CRISPR-Cas12a is LbCas12a.

In yet another embodiment, the CHIP further comprises an anthocyanin marker. The anthocyanin marker is selected from the group consisting of R1-navajo and R1-SCM2. In one embodiment, the anthocyanin marker is R1-navajo and in another embodiment, the anthocyanin marker is R1-SCM2. The anthocyanin marker is homozygous. In yet another embodiment, the CHIP further comprises a restorer allele, wherein the restorer allele is selected from the group consisting of Rf3, Rf4, Rf11, Rf10 and Rf12. In an embodiment, the restorer allele is Rf4. In another embodiment, the restorer allele is homozygous. In another embodiment, the CHIP comprises a non-restorer allele, wherein the non-restorer allele is selected from the group consisting of rf3, rf4, rf10, rf11, and rf12. The non-restorer allele is rf4 and homozygous.

In an embodiment, the CHIP comprises a cenh3 mutation, a R1-navajo marker, and a restorer allele of a restorer factor 4 gene. In another embodiment, the CHIP comprises a cenh3 mutation, a R1-SCM2 marker, and a restorer allele of a restorer factor 4 gene. In one embodiment, the CHIP comprises a cenh3 mutation, a R1-navajo marker, and a non-restorer allele of a restorer factor 4 gene while in another embodiment, the CHIP comprises a cenh3 mutation, a R1-SCM2 marker, and a non-restorer allele of a restorer factor 4 gene. In yet another embodiment, the CHIP is selected from the group consisting of maize, wheat, rice, sunflower, tomato, barley, brassicas, cucumber, and watermelon. In another embodiment, the CHIP is maize.

In an embodiment, the DIP is a pollen donor in the cross of the CHIP and DIP. The DIP may be homozygous or heterozygous for a non-restorer allele. In another embodiment, the DIP is homozygous for the non-restorer allele, wherein the non-restorer allele is selected from the group consisting of rf3, rf4, rf10, rf11, and rf12. In one embodiment, the non-restorer allele is rf4. In an embodiment, the DIP is selected from the group consisting of maize, wheat, rice, and sunflower, tomato, barley, brassicas, cucumber, and watermelon. In one embodiment, the DIP is maize.

In yet another embodiment, disclosed herein is a plant produced by the method described above, wherein the plant is a CMS haploid plant. In an embodiment, the CMS haploid plant comprises the CMS cytoplasm of the CHIP and the nuclear genome of the DIP while lacking an anthocyanin marker, a restorer allele, and a cenh3 knockout mutation. In another embodiment, the CMS haploid plant is treated with a doubling agent. In one embodiment, the doubling agent is selected from the group consisting of colchicine, pronamide, dithipyr, trifluralin, nitrous oxide, or another known anti-microtubule agent. In another embodiment, the doubling agent is colchicine. In yet another embodiment, the CMS haploid plant is pollinated with pollen from the DIP. In another embodiment, the CMS haploid plant is pollinated with preserved pollen. In yet another embodiment, the CMS haploid plant is confirmed CMS by genotyping or other molecular analysis.

### EXAMPLES

### Example 1: Creating a CMS Haploid Inducer Line (CHIP) with the R1-Nj Color Marker

1. To select efficient gRNAs for gene-editing, we transfected etiolated maize protoplasts with LbCas12a-crRNA RNP complexes with various candidate gRNAs. The crRNA scaffold used for LbCas12a is based on the CRISPR-LbCpf1 system. Protoplasts were isolated from etiolated maize leaves grown under dark conditions as described (Sheen, 1991). Protoplast transfection was carried out as described (Sant′Ana et al., 2020) with some modifications. Transfection reactions consisted of 5×10⁵ protoplasts per reaction and were incubated with PEG solution (40% PEG-4000, 0.6M Mannitol, 100 mM CaCl2) for 15 min. Following termination by W5 solution (154 mM NaCl, 125 mM CaCl2, 5 mM KCl and 2 mM MES, pH 5.7), transfected protoplasts were resuspended in 300 ulW1 solution (0.6M Mannitol, 4 mM MES, pH 5.7, 4 mM KCl), transferred to 96-well clear bottom microplate and incubated for 2 days in the dark at 28° C. without shaking. DNA was isolated from transfected protoplasts 2 days later and analyzed for gene-editing efficiency by PCR amplification followed by restriction of the amplicons with T7 endonuclease I (NEB.)

2. Biolistic bombardment of SYN-INBC34 inbred immature embryos was performed using LbCas12a RNP carrying gRNA140, selected as described above, with sequence CAGGTGGTGCGAGTACCTCGGCG (SEQ ID NO: 1), targeting the second exon of gene ID GRMZM2G158526, and the DNA vector 26258 (see Table 16) which carries a PMI selectable marker. To generate LbCas12a-crRNA RNP complexes, 0.3 nmol of Cas12 protein and 0.3 nmol of crRNA were mixed in a total volume of 11 μl and incubated at room temperature for 10 minutes. For RNP delivery alone, the RNPs were coated onto 0.6 μm gold particles (Bio-Rad, USA) as follows: 100 μl of gold particles (water suspension of 10 mg/ml) and 20 μl of glycogen (20 mg/ml) were added to premixed RNPs, mixed gently, and then incubated on ice for 10 minutes. For co-delivery of RNP with DNA, the RNPs and DNA vector plasmid 26258 were coated onto gold particles as follows: 100 μl of gold particles (water suspension of 10 mg/ml) and 20 μl of glycogen (20 mg/ml) were added to premixed RNPs and DNA vector, mixed gently, and incubated on ice for 10 minutes. The RNP/DNA coated gold particles were centrifuged at 8,000 g for 40 seconds and the supernatant removed. The pellet was resuspended with 30 μl of sterile water by brief sonication, and then spread onto a macro-carrier disc (10 μl each) followed by air dry in the laminar flow hood (2-4 h).

3. Immature embryos were isolated from harvested ears about 9-11 days after pollination and pre-cultured for 1-3 days on osmoticum media. Pre-cultured embryos were then bombarded with the LbCas12a-RNP complex and DNA described above using the BioRad PDS-1000 HeTM Biolistic particle delivery system. Bombarded embryos were incubated in callus induction media. Induced calli were then moved onto mannose selection media. Mannose resistant calli were transferred to regeneration media to induce shoot formation. Shoots were then sub-cultured onto rooting media. Leaf samples were harvested from rooted plants for TaqMan R assays to detect mutations in the target site using a previously described real time quantitative polymerase chain reaction (qPCR) TaqMan R method.

4. We positively identified CenH3-het edited plants using TaqMan assay 3895 with primers TCCTTGTTCCGTCTTTTGCAG (SEQ ID NO: 2) and AAGGCAAAAGGAGGGAACTGAT (SEQ ID NO: 3), and probe TACCTCGGCGACGCC (SEQ ID NO: 4), and for frameshift alleles by PCR-sequencing. SYN-INBC34 is rf4/rf4, does not have the R-nj marker, and is not CMS. The following steps were made to bring the edited CenH3 allele into a genetic background that comprised homozygous R1-nj, CMS, and homozygous Rf4.

5. TO events were grown to maturity and self-pollinated and/or outcrossed as males onto a CMS-C material, SYN-INB77M-CMS (rf4/rf4 and male sterile). T1 generation plants from TO selfing were also identified by TaqMan assay and PCR-sequencing as being heterozygous for frameshift mutations in the CenH3 coding sequence (from here on referred to as CenH3 [+/−]plants) and grown to maturity for crossing onto CMS to generate more CMS-Cenh3 edited seeds.
a. In the F1 generation after crossing TO or T1 CenH3 mutant pollen onto SYN-INB77M-CMS, we identified a plant carrying a heterozygous 10 base deletion in CENH3 by TaqMan assay and Sanger sequencing. This plant was male sterile and pollinated by pollen from a RWKS plant line homozygous for R-nj and Rf4.
b. T1 generation (non-CMS) CenH3 (+/−) mutant plants were also reciprocally crossed to RWKS to generate F1s with CenH3 and the R-nj marker.
c. All seeds from the CMS CenH3 (+/−) x RWKS cross from step 5b were red or purple, indicating the R1-nj marker was present in at least a heterozygous condition. The seeds are planted and the resulting plants genotyped for the CenH3 (+/−) and Rf4 (+/−) zygosity by TaqMan assay and then self-pollinated (they are fertile due to Rf4 marker). Some of the plants are also backcrossed by seed carrying CenH3 (+/−) and R-nj from F1 progeny of the CenH3 (+/−) x RWKS cross in step 5b.
d. Purple seeds resulting from the crosses in step 5c are planted and selected by TaqMan and sequencing for CenH3 (+/−) heterozygous, R-nj homozygous, and CMS cytoplasm. These inducer materials are easily increased by selfing. If the Rf4 marker is not present (i.e., the inducer plants are rf4/rf4), one may also use crossing to a sibling or maintainer plant carrying the R-nj marker and optionally a mutant allele of CenH3 (+/+ or +/−) to increase the seed.
e. This inducer is then used as a CMS donor line for one-step conversions. The material is used as a female and crossed by pollen from any line that is desired to be converted directly to CMS cytoplasm. After the cross, haploids are color sorted as mature, dry seed having cream-colored embryos (the diploid hybrids have purple embryos). Then, the haploid seed may or may not be chemically treated to induce genome doubling prior to planting in soil and being grown to maturity where they are then crossed by recurrent parent pollen.

TABLE 1
Nucleotide alignment of CENH3 deletion mutant alleles compared to the wild type
(SEQ ID NO: 7) (509A115A represented by SEQ ID NO: 8; 509A151A represented by
SEQ ID NO: 9, and 509A150A represented by SEQ ID NO: 177)

| | |
|---|---|
| GRMZM2G158526 | CTCTCCGACCCTGGTGCTAAGCACGTTCCTTGTTCCGTCTTTTGCAGGTGGTGCG |
| 509A115A | CTCTCCGACCCTGGTGCTAAGCACGTTCCTTGTTCCGTCTTTTGCAGGTGGTGCG |
| 509A150A | CTCTCCGACCCTGGTGCTAANCACGTTCCTTGTTCCGTCTTTTGCAGGTGGTGCG |
| 509A151A | CTCTCCGACCCTGGTGCTAAGCACGTTCCTTGTTCCGTCTTTTGCAGGTGGTGCG |
| GRMZM2G158526 | AGTACCTCGGCGACGCCGGTGAGCGCGTGCGTGCGGGGATCAGTTCCCTCCTTTT |
| 509A115A | AG----------ACGCCGGTGAGCGCGTGCGTGCGGGGATCAGTTCCCTCCTTTT |
| 509A150A | AG-------------------AGCGCGTGCGTGCGGGGATCAGTTCCCTCCTTTT |
| 509A151A | -------------------TGAGCGCGTGCGTGCGGGGATCAGTTCCCTCCTTTT |
| GRMZM2G158526 | GCCTTTTTTTGTTGGGCTGCTCTTACTTGCTTGCAAGCTGTTTGATGGAATGCAG |
| 509A115A | GCCTTTTTT-GT-GGGCTGCTCTTACTTGCTTGCAAGCTGTTTGATGGAATGCAG |
| 509A150A | GCCTTTTTT-GT-GGGCTGCTCTTACTTGCTTGCAAGCTGTTTGATGGAATGCAG |
| 509A151A | GCCTTTTTT-GT-GGGCTGCTCTTACTTGCTTGCAAGCTGTTTGATGGAATGCAG |
| GRMZM2G158526 | GAAAGGGCTGCTGGGACCGGGGGAAG |
| 509A115A | GAAAGGGCTGCTGGGACCGGGGGAAG |
| 509A150A | GAAAGGGCTGCTGGGACCGGGGGAAG |
| 509A151A | GAAAGGGCTGCTGGGACCGGGGGAAG |

TABLE 2
Partial alignment of deduced amino acid sequences of CENH3 deletion mutant
alleles (GRMZM2G158526 WT represented by SEQ ID NO: 10). The 509A115A (10 bp
deletion SEQ ID NO: 12) occurs entirely in exon 2 so the
deduced splicing was kept as in the wild type. For 509A151A and 509A150A
(the 19 bp deletions), we generated all possible splice variants
(SEQ ID NOs: 13-22) depending on the GT donor site used for
intron splicing. The deduced amino acid sequence for the KD allele (Kelly Dawe,
University of Georgia) is also shown (SEQ ID NO: 11).

| | |
|---|---|
| GRMZM2G158526 | MARTKHQAVRKTAEKPKKKLQFERSGGASTSATP--------ERAAG--- |
| CENH3_KD | MARTKHQAVRKTAEKPKKKLQFERSGGASTSATP--------ERAAG--- |
| 509A115A_transcript_ | MARTKHQAVRKTAEKPKKKLQFERSGGARRR------------------- |
| 509A151A_transcript_a | MARTKHQAVRKTAEKPKKKLQFERSGGASTSATPVSA------------- |
| 509A151A_transcript_b | MARTKHQAVRKTAEKPKKKLQFERSGGASTSATPVSAC----ERAAG--- |
| 509A151A_transcript_c | MARTKHQAVRKTAEKPKKKLQFERSGGASTSATPVSACVRGSERAAG--- |
| 509A151A_transcript_d | MARTKHQAVRKTAEKPKKKLQFERSGGASTSATPVSACVRGSVPSFCLFL |
| 509A151A_transcript_e | MARTKHQAVRKTAEKPKKKLQFERSGGASTSATPVSACVRGSVPSFCLFL |
| 509A150A_transcript_a | MARTKHQAVRKTAEKPKKKLQFERSGGASTSATPVSA------------- |
| 509A150A_transcript_b | MARTKHQAVRKTAEKPKKKLQFERSGGASTSATPVSAC----ERAAG--- |
| 509A150A_transcript_c | MARTKHQAVRKTAEKPKKKLQFERSGGASTSATPVSACVRGSERAAG--- |
| 509A150A_transcript_d | MARTKHQAVRKTAEKPKKKLQFERSGGASTSATPVSACVRGSVPSFCLFL |
| 509A150A_transcript_e | MARTKHOAVRKTAEKPKKKLQFERSGGASTSATPVSACVRGSVPSFCLFL |
| GRMZM2G158526 | TG---------GRAASGGDSVKKTKPRHRWRPGTVALREIRKYQKSTEPL |
| CENH3_KD | TG---------GRAASGGDSVKKTKPRHRWRPGL*RCGRSGSTRSPLNRS |
| 509A115A_transcript_ | KG---LL----GPGEERRLEVTQLRRRNHATAGGQGL*RCGRSGSTRSPL |
| 509A151A_transcript_a | KG---LL----GPGEERRLEVTQLRRRNHATAGGQGL*RCGRSGSTRSPL |
| 509A151A_transcript_b | TG---------GRAASGGDSVKKTKPRHRWRPGTVALREIRKYQKSTEPL |
| 509A151A_transcript_c | TG---------GRAASGGDSVKKTKPRHRWRPGTVALREIRKYQKSTEPL |
| 509A151A_transcript_d | KG---LL----GPGEERRLEVTQLRRRNHATAGGQGL*RCGRSGSTRSPL |
| 509A151A_transcript_e | LGCSYLLAS*KGCWDRGKSGVWR*LS*EDETTPPLAARDCSAAGDQEVPE |
| 509A150A_transcript_a | KG---LL----GPGEERRLEVTQLRRRNHATAGGQGL*RCGRSGSTRSPL |
| 509A150A_transcript_b | TG---------GRAASGGDSVKKTKPRHRWRPGTVALREIRKYQKSTEPL |
| 509A150A_transcript_c | TG---------GRAASGGDSVKKTKPRHRWRPGTVALREIRKYQKSTEPL |
| 509A150A_transcript_d | KG---LL----GPGEERRLEVTQLRRRNHATAGGQGL*RCGRSGSTRSPL |
| 509A150A_transcript_e | LGCSYLLAS*KGCWDRGKSGVWR*LS*EDETTPPLAARDCSAAGDQEVPE |
| GRMZM2G158526 | IPFAPF-VRVVR---ELTNFVTN--GKVERYTAEALLALQE |
| CENH3_KD | *GS*PIS*QTGK*SAIPQKPSLRC-K-S---PLRLSSVW-- |
| 509A115A_transcript_ | NRSSPLRLSSVW---*GS*PIS*QTGK*SAIPQKPSLRCK- |
| 509A151A_transcript_a | NRSSPLRLSSVW---*GS*PIS*QTGK*SAIPQKPSLRCK- |
| 509A151A_transcript_b | IPFAPF-VRVVR---ELTNFVTN--GKVERYTAEALLALQE |
| 509A151A_transcript_c | IPFAPF-VRVVR---ELTNFVTN--GKVERYTAEALLALQE |
| 509A151A_transcript_d | NRSSPLRLSSVW---*GS*PIS*QTGK*SAIPQKPSLRCK- |
| 509A151A_transcript_e | VH*TAHPLCAFRPCGEGVNQFRNKRESRALYRRSPPCAAR- |
| 509A150A_transcript_a | NRSSPLRLSSVW---*GS*PIS*QTGK*SAIPQKPSLRCK- |
| 509A150A_transcript_b | IPFAPF-VRVVR---ELTNFVTN--GKVERYTAEALLALQE |
| 509A150A_transcript_c | IPFAPF-VRVVR---ELTNFVTN--GKVERYTAEALLALQE |
| 509A150A_transcript_d | NRSSPLRLSSVW---*GS*PIS*QTGK*SAIPQKPSLRCK- |
| 509A150A_transcript_e | VH*TAHPLCAFRPCGEGVNOFRNKRESRALYRRSPPCAAR- |

### Example 2: Creating a CMS Haploid Inducer Line (“CHIP”) with the R1-SCM2 Color Marker

1. To select efficient gRNAs for gene-editing we transfected etiolated maize protoplasts with LbCas12a-crRNA RNP complexes with various candidate gRNAs. The crRNA scaffold used for LbCas12a is based on the CRISPR-LbCpf1 system. Protoplasts were isolated from etiolated maize leaves grown under dark conditions as described (Sheen, 1991). Protoplast transfection was carried out as described (Sant′Ana et al., 2020) with some modifications. Transfection reactions consisted of 5×10⁵ protoplasts per reaction and were incubated with PEG solution (40% PEG-4000, 0.6M Mannitol, 100 mM CaCl2) for 15 minutes. Following termination by W5 solution (154 mM NaCl, 125 mM CaCl2, 5 mM KCl and 2 mM MES, pH 5.7), transfected protoplasts were resuspended in 300 μl W1 solution (0.6M Mannitol, 4 mM MES, pH 5.7, 4 mM KCl), transferred to 96-well clear bottom microplate, and incubated for 2 days in the dark at 28° C. without shaking. DNA was isolated from transfected protoplasts after 2 days and analyzed for gene-editing efficiency by PCR amplification followed by restriction of the amplicons with T7 endonuclease I (NEB.)
2. Biolistic bombardment of SYN-INBC34 x SYN-INBC34RS isolated immature embryos (heterozygous for the R1-SCM2 marker) was performed using LbCas12a complexed with gRNA140 (sequence CAGGTGGTGCGAGTACCTCGGCG, SEQ ID NO: 1), targeting the second exon of gene ID GRMZM2G158526, and the DNA vector 26258 (Table 16), both of which carry a PMI selectable marker. To generate Cas12a-crRNA RNP complexes, 0.3 nmol of Cas12 protein and 0.3 nmol of crRNA were mixed in a total volume of 11 μl and incubated at room temperature for 10 minutes. For RNP delivery alone, the RNPs were coated onto 0.6 μm gold particles (Bio-Rad, USA) as follows: 100 μl of gold particles (water suspension of 10 mg/ml) and 20 μl of glycogen (20 mg/ml) were added to premixed RNPs, mixed gently, and incubated on ice for 10 minutes. For co-delivery of RNP with DNA, the RNPs and DNA vector plasmid 26258 were coated onto gold particles as follows: 100 μl of gold particles (water suspension of 10 mg/ml) and 20 μl of glycogen (20 mg/ml) were added to premixed RNPs and DNA vector, mixed gently, and incubated on ice for 10 minutes. The RNP/DNA coated gold particles were centrifuged at 8,000 g for 40 s and the supernatant removed. The pellet was resuspended with 30 μl of sterile water by brief sonication and spread onto a macro-carrier disc (10 μl each) followed by air dry in the laminar flow hood (2-4 h).
3. Immature embryos were isolated from harvested ears about 9-11 days after pollination and pre-cultured for 1-3 days on osmoticum media. Pre-cultured embryos were bombarded with the LbCas12a-RNP complex and DNA described above using the BioRad PDS-1000 HeTM Biolistic particle delivery system. Bombarded embryos were then incubated in callus induction media. Induced calli were moved onto mannose selection media. Mannose resistant calli were transferred to regeneration media to induce shoot formation. Shoots were then sub-cultured onto rooting media. Leaf samples were harvested from rooted plants for TaqMan R assays to detect mutations in the target site using a previously described real time quantitative polymerase chain reaction (qPCR) TaqMan R method.
4. We positively identified CenH3-het edited plants using TaqMan assay 3895, using primers TCCTTGTTCCGTCTTTTGCAG (SEQ ID NO: 2) and AAGGCAAAAGGAGGGAACTGAT (SEQ ID NO: 3) and probe TACCTCGGCGACGCC (SEQ ID NO: 4) and for frameshift alleles by PCR-sequencing. The following steps were made to bring the edited CenH3 allele into a genetic background that comprised homozygous R1-SCM2, CMS, and homozygous Rf4.
5. TO events were grown to maturity and selfed. Purple seeded T1 progeny were planted and CenH3 (+/−) plants were identified by TaqMan and PCR-sequencing and grown to maturity for selfing and crossing onto SYN-INB77M-CMS line ears, which carry the maintainer alleles rf4 and rf11.
5 6. Two SYN-INB77M-CMS ears were obtained after pollinations with plants carrying the R1-SCM2 marker and a 19 base pair deletion of sequence TACCTCGGCGACGCCGGTG (SEQ ID NO: 5). The progeny seed to be planted are selected again for purple seed color. Seedlings are genotyped and further selected for homozygous R1-SCM2 using R1 markers and heterozygous for the 19 bp deletion with a TaqMan assay specific for this cenh3 mutant allele. If the plants are male fertile, they are selfed. If the plants are male sterile, they are crossed as females by non-CMS T2 plants with R1-SCM2 and CenH3 (+/−) present.

TABLE 3
List of markers for identification of desired genotypes of R1 color marker and R1-color inhibitor.

| | | | | | | | Embryo/ |
|---|---|---|---|---|---|---|---|
| Marker | SM0954BQ | SM6623 | SM6568 | SM0953BQ | SM8040 | SM8091 | Seed Color |
| Chromosome | 10 | | | | 9 | | N/A |
| Trait/QTL/Gene | R1-SCM2 dominant color inducer | | | | Color Inhibitor | | N/A |
| Location (B73v5) | 139300360 | 139789157 | 139789170 | 140146379 | 8120045 | 10040065 | N/A |
| RWKS (contains | C/C | G/G | T/T | A/A | G/G | C/C | Dark Purple |
| R1-SCM2) | | | | | | | |
| RWK (contains | C/C | G/G | T/T | A/A | | A/A | Light Purple |
| R-Navajo) | | | | | | | |
| SYN-INBC34 | A/A | A/A | A/A | G/G | A/A | A/A | Cream/Yellow |
| SYN-INB77M-CMS | C/C | A/A | A/A | A/A | | | Cream/Yellow |

TABLE 4
List of markers to identify the type of cytoplasm or mitochondrial genome.

| | | | | | Cytotype |
|---|---|---|---|---|---|
| Marker | SM2918 | SM4813 | SM2914 | SM4812 | call |
| Chromosome | Mitochondrial Genome | | | | |
| RWKS | C/C | C/C | A/A | D/D | Normal B |
| RWK | C/C | C/C | A/A | D/D | Normal B |
| SYN-INBC34 | C/C | C/C | C/C | I/I | Normal A |
| SYN-INB77M-CMS | A/A | A/A | C/C | I/I | CMS |

TABLE 5
List of markers for identification of type of restorer gene alleles.

| | | | | | Restorer Factor |
|---|---|---|---|---|---|
| Marker | SM2669 (Rf4) | SM2670 (Rf4) | SM7200 (Rf11) | SM5665 (Rf11) | Phenotype call |
| Chromosome | 8 | | 2 | | |
| RWKS | G/G | I/I | C/C | G/G | Rf4/Rf11 |
| | | | | | (restorer) |
| RWK | G/G | I/I | C/C | G/G | Rf4/Rf11 |
| | | | | | (restorer) |
| SYN-INBC34 | A/A | D/D | C/C | G/G | rf4/Rf11 |
| | | | | | (depends) |
| SYN-INB77M-CMS | A/A | D/D | G/G | C/C | rf4/rf11 |
| | | | | | (maintainer) |

TABLE 6
RT-PCR sequences for CENH3 assays.

| ZmCE | | | | |
|---|---|---|---|---|
| NH3 | Assay | | | |
| version | Name | Primer 1 | Primer 2 | Probe |
| wild | PM1 | AAGGCAAAAGGAGGG | TCCTTGTTCCGTCTTT | TACCTCGGCG |
| type | 901 | AACTGAT (SEQ ID | TGCAG (SEQ ID NO: 99) | ACGCC (SEQ |
| | | NO: 98) | | ID NO: 100) |
| 10 bp | PM1 | TCCTTGTTCCGTCTTT | AAGGCAAAAGGAGGG | CCGGCGTCTC |
| deletion | 909 | TGCAG (SEQ ID NO: | AACTGAT (SEQ ID NO: | GC (SEQ ID NO: |
| event | | 101) | 102) | 103) |
| 509A115A | | | | |
| 19 bp | PM1 | TCCTTGTTCCGTCTTT | AAGGCAAAAGGAGGG | CGCGCTCACG |
| deletion | 913 | TGCAG (SEQ ID NO: | AACTGAT (SEQ ID NO: | CAC (SEQ ID |
| in event | | 101) | 102) | NO: 104) |
| 509A151A | | | | |

7. The next generation is genotyped for homozygosity of R1-SCM2, the presence of CMS, and the heterozygous knockout CenH3 (+/−) configuration. This specific combination is one of the ideal inducer genotypes that can be used for CMS cyto-swapping. These inducer materials are easily increased by selfing. However, if the line is male sterile (i.e., the Rf4 marker is not present and the inducer plants are rf4/rf4), one may also cross these inducers as females by maintainer pollen, which are homozygous for the R1-SCM2 or R-nj marker and optionally a mutant allele of CenH3 (+/+ or +/−) to increase the seed.
8. This inducer is then used as a CMS donor line for one-step conversions. The material is used as a female and crossed by pollen from any line that is desired to be converted directly to CMS cytoplasm. After the cross, haploids are color sorted between 10 and 25 days after pollination in vitro (we select the cream-colored embryos after 24 hours of color induction in an incubator). The haploid seed may or may not be chemically treated to induce genome doubling and simply transplanted to soil and grown for further crossing by the recurrent parent. Again, fertile inducer plants may be maintained by selfing while sterile inducers can be crossed to a non-CMS (i.e., normal cytoplasm) maintainer line carrying the R1-SCM2 (or R-nj) color marker. Optionally, the maintainer line may have the non-restorer alleles of rf4 and rf11 as well as CenH3 WT or CenH3 knockout (mutant) alleles.

### Example 3. Cyto-Swapping Proof of Concept in Maize (Normal a Cytoswap)

Cyto-swapping in maize using a CenH3 (+/−) inducer was demonstrated by obtaining genome edited material from the laboratory of Kelly Dawe, in the Department of Plant Biology at the University of Georgia, and crossing it to a panel of maize lines to generate haploids. The haploids were then doubled and selfed to create DH1 seeds. The cytoplasm of the material acquired from Dr. Dawe was “Normal A”, a cytoplasm known to be common to transformable maize genetic backgrounds and features characteristic genotypes for the SM2914 marker. In contrast, the line selected to cyto-swap to this Normal A were known to be Normal B, which is not a transformable background and features distinct characteristic genotypes for the SM2914 marker.

We genotyped the plants from Dr. Dawe for the CenH3 edited allele, selected CenH3 heterozygous (+/−) plants that were CenH3 heterozygous (+/−), and crossed those as females to seven Normal B Syngenta males. We harvested the ears 16 days after pollination and isolated embryos onto germination medium in petri plates containing colchicine for 24 hours to induce genome doubling. We then moved the embryos to a recovery medium in phytatrays and grew the plants to the two-leaf stage. Then, we sampled the leaves from the plants and extracted DNA for evaluation with a panel of TaqMan endpoint markers that were polymorphic between the Normal A haploid inducer and the Normal B male parents. These markers cover all 10 chromosomes and were used to select plants that only carry chromosomes from the pollen donor.

TABLE 7
Panel of markers for identification of haploid plants

| | SEQ ID NOs for | SEQ ID NOs for | SEQ ID NOs for | SEQ ID NOs for | SEQ ID NOs for |
|---|---|---|---|---|---|
| Assay Name | F primer | R primer | probe 1 | probe 2 | Target Sequence |
| SM0253EQ | 23 | 24 | 25 | 26 | 27 |
| SM0093B | 28 | 29 | 30 | 31 | 32 |
| SM0435A | 33 | 34 | 35 | 36 | 37 |
| SM1071CQ | 38 | 39 | 40 | 41 | 42 |
| SM1280CQ | 43 | 44 | 45 | 46 | 47 |
| SM1447AQ | 48 | 49 | 50 | 51 | 52 |
| SM1847AQ | 53 | 54 | 55 | 56 | 57 |
| SM1286AQ | 58 | 59 | 60 | 61 | 62 |
| SM2513 | 63 | 64 | 65 | 66 | 67 |
| SM2962 | 68 | 69 | 70 | 71 | 72 |
| SM2988 | 73 | 74 | 75 | 76 | 77 |
| SM3400 | 78 | 79 | 80 | 81 | 82 |
| SM3747 | 83 | 84 | 85 | 86 | 87 |
| SM4788 | 88 | 89 | 90 | 91 | 92 |
| SM5515 | 93 | 94 | 95 | 96 | 97 |

We identified “paternal haploid” offspring by virtue of their being “homozygous” for the male parent's markers and sorted those plants from those genotyped as hybrids which were heterozygous for all nuclear DNA markers in the panel. Table 8 below shows the summary for the haploid induction rate based on this data. Several plants were also tested by ploidy analysis to determine if they were indeed haploids. See

FIGS. 3a (diploid control) and 3b (haploid).

TABLE 8
Haploid induction rate summary for paternal haploid offspring.

| | Embryos | Plantlets | Survival | Haploids | HI Rate | Survived to |
|---|---|---|---|---|---|---|
| Line | extracted | genotyped | rate | selected | (%) | maturity |
| SYN-INB51B | 322 | 235 | 73.0 | 10 | 4.3 | 10 |
| SYN-INB82Q | 286 | 241 | 84.3 | 24 | 10.0 | 23 |
| SYN-INB16S | 532 | 339 | 63.7 | 25 | 7.4 | 24 |
| SYN-INB93A | 169 | 136 | 80.5 | 12 | 8.8 | 10 |
| SYN-INB78V | 368 | 274 | 74.5 | 9 | 3.3 | 8 |
| SYN-INB40F | 84 | 59 | 70.2 | 6 | 10.2 | 6 |
| SYN-INB25Y | 373 | 188 | 50.4 | 10 | 5.3 | 10 |

TABLE 9
Cyto-swapping proof of concept and efficiency
test in diverse maize germplasm. Confirmation
of cytoplasm swapping in Doubled Haploids

| Plant Unique Id | Cytotype | Allele Call by SM2914 |
|---|---|---|
| SYN-INB82Q | Nb | A/A |
| SYN-INB82Q | Nb | A/A |
| SYN-INB82Q | Nb | A/A |
| SYN-INB82Q | Nb | A/A |
| SYN-INBAD33 | Na | C/C |
| SYN-INBAD33 | Na | C/C |
| SYN-INBAD33 | Na | C/C |
| SYN-INBAD33 | Na | C/C |
| UR264309735 | Na | C/C |
| UR264309752 | Na | C/C |
| UR264309759 | Na | C/C |
| UR264309776 | Na | C/C |
| UR264308770 | Na | C/C |
| UR264308079 | Na | C/C |
| UR264308078 | Na | C/C |
| UR264308071 | Na | C/C |
| UR264308049 | Na | C/C |
| UR264308042 | Na | C/C |
| UR264308034 | Na | C/C |
| UR264307985 | Na | C/C |
| UR264307984 | Na | C/C |
| UR264307977 | Na | C/C |
| UR264307971 | Na | C/C |
| UR264307967 | Na | C/C |
| UR264307946 | Na | C/C |
| UR264307935 | Na | C/C |
| UR264307912 | Na | C/C |
| UR264307898 | Na | C/C |
| UR264307897 | Na | C/C |
| UR264307788 | Na | C/C |
| UR264307785 | Na | C/C |
| UR264307782 | Na | C/C |
| UR264307764 | Na | C/C |
| UR264307760 | Na | C/C |
| UR264307755 | Na | C/C |
| UR264307754 | Na | C/C |
| UR264307707 | Na | C/C |
| UR264307669 | Na | C/C |
| UR264308788 | Na | C/C |
| UR264308835 | Na | C/C |
| UR264308864 | Na | C/C |
| UR264308995 | Na | C/C |
| UR264309001 | Na | C/C |
| UR264308766 | Na | C/C |
| UR264308760 | Na | C/C |
| UR264308751 | Na | C/C |
| UR264308750 | Na | C/C |
| UR264308537 | Na | C/C |
| UR264308533 | Na | C/C |
| UR264308456 | Na | C/C |
| UR264308427 | Na | C/C |
| UR264308426 | Na | C/C |
| UR264308402 | Na | C/C |
| UR264308358 | Na | C/C |
| UR264308357 | Na | C/C |
| UR264308213 | Na | C/C |
| UR264308143 | Na | C/C |
| UR264308142 | Na | C/C |
| UR264308129 | Na | C/C |
| UR264308117 | Na | C/C |
| UR264308083 | Na | C/C |
| UR264309329 | Na | ND |
| UR264309333 | Na | C/C |
| UR264309334 | Na | C/C |
| UR264309337 | Na | C/C |
| UR264309338 | Na | C/C |
| UR264309340 | Na | C/C |
| UR264309343 | Na | C/C |
| UR264309401 | Na | C/C |
| UR264309414 | Na | C/C |
| UR264309415 | Na | C/C |
| UR264309416 | Na | C/C |
| UR264309417 | Na | C/C |
| UR264309425 | Na | C/C |
| UR264309432 | Na | C/C |
| UR264309567 | Na | C/C |
| UR264309598 | Na | C/C |
| UR264309605 | Na | C/C |
| UR264309624 | Na | C/C |
| UR264309051 | Na | C/C |
| UR264309053 | Na | C/C |
| UR264309054 | Na | C/C |
| UR264309056 | Na | C/C |
| UR264309057 | Na | C/C |
| UR264309061 | Na | C/C |
| UR264309064 | Na | C/C |
| UR264309085 | Na | C/C |
| UR264309087 | Na | C/C |
| UR264309090 | Na | C/C |
| UR264309142 | Na | C/C |
| UR264309260 | Na | C/C |
| UR264309265 | Na | C/C |
| UR264309325 | Na | C/C |
| UR264309326 | Na | C/C |
| UR264309328 | Na | C/C |
| UR264309632 | Na | C/C |

The data shows we recovered between 3 and 10% haploids from the inducer crosses. The haploids were grown to maturity and self-pollinated (recall that they had been genome doubled via colchicine treatment, so it is more reasonable to say that they were doubled haploids). The seed numbers shown below in Table 10 were obtained on the self-pollinated ears.

TABLE 10
Seed produced from selfed cytoplasm-converted Doubled Haploids

| MATID | MAT:FPPSS:BARCD | Seed quantity |
|---|---|---|
| 21SBI227842 | UR264309001 | 293 |
| 21SBI227825 | UR264308750 | 334 |
| 21SBI227809 | UR264308760 | 207 |
| 21SBI227866 | UR264308766 | 321 |
| 21SBI227822 | UR264308788 | 372 |
| 21SBI227820 | UR264308357 | 117 |
| 21SBI227823 | UR264308402 | 73 |
| 21SBI227819 | UR264308426 | 277 |
| 21SBI227813 | UR264308427 | 33 |
| 21SBI227807 | UR264308071 | 80 |
| 21SBI227824 | UR264308078 | 146 |
| 21SBI227836 | UR264308117 | 244 |
| 21SBI227826 | UR264308143 | 288 |
| 21SBI228173 | UR264307897 | 298 |
| 21SBI227830 | UR264307935 | 238 |
| 21SBI228174 | UR264307967 | 393 |
| 21SBI228176 | UR264307971 | 292 |
| 21SBI228170 | UR264307977 | 310 |
| 21SBI227815 | UR264307985 | 158 |
| 21SBI227806 | UR264307754 | 10 |
| 21SBI227209 | UR264307755 | 122 |
| 21SBI227821 | UR264307764 | 5 |
| 21SBI227844 | UR264309051 | 267 |
| 21SBI227817 | UR264309053 | 17 |
| 21SBI227837 | UR264309054 | 245 |
| 21SBI227827 | UR264309056 | 354 |
| 21SBI227846 | UR264309057 | 285 |
| 21SBI227864 | UR264309061 | 151 |
| 21SBI227865 | UR264309064 | 262 |
| 21SBI227840 | UR264309085 | 272 |
| 21SBI227871 | UR264309090 | 376 |
| 21SBI227873 | UR264309142 | 281 |
| 21SBI227868 | UR264309265 | 176 |
| 21SBI228227 | UR264309333 | 96 |
| 21SBI227860 | UR264309401 | 14 |
| 21SBI228214 | UR264309415 | 185 |
| 21SBI228190 | UR264309417 | 50 |
| 21SBI228186 | UR264309425 | 289 |
| 21SBI227808 | UR264309567 | 217 |
| 21SBI227839 | UR264309605 | 157 |
| 21SBI227848 | UR264309624 | 5 |
| 21SBI227863 | UR264309632 | 22 |
| 21SBI227858 | UR264309735 | 30 |
| 21SBI227867 | UR264309752 | 5 |
| 21SBI227862 | UR264309759 | 27 |
| 21SBI227861 | UR264309776 | 12 |

Importantly, both the doubled haploids and the DH1 generation offspring were genotyped and shown to have the cytoplasm markers associated with Normal A, not Normal B. This clearly indicates that the cyto-swapping concept works efficiently using a CenH3+/− inducer. This concept is easily applied to swapping male sterile cytoplasm (shown in the examples below).

### Example 4: CMS Cyto-Swapping Recurrent Parents Using Heterozygous CENH3 Lines and the R1-SCM2 Marker

Step 1. We selected thirteen DIP corn lines (9 field corn and 4 sweet corn) for converting to CMS. These lines possess the non-fertility restoration genotypes rf4 and rf11. DIP corn lines may also be referred to as Recurrent Parents.
Step 2. We planted seed of two kinds of conversion line materials, one segregating for a 19 bp deletion in CENH3 (SEQ ID NO: 9) and another segregating for a 10 bp deletion in CENH3 (SEQ ID NO: 8). Both types of materials segregate wild type 1:1 heterozygous and carry the R1-SCM2 allele (an anthocyanin marker which expresses in the scutellum of an embryo).
Step 3. From these plantings, we identified the cenh3 mutant heterozygous plants using TaqMan assays specific for the wild type and both deletion alleles (3 assays total) and grew them to maturity. These resulting lines are the conversion line plants, i.e., CHIP plants.

TABLE 11
Assays used to identify cenh3 heterozygous plants, type of cytoplasm
(mitochondrial genome) and desired alleles at R1, C1, Rf4, and Rf11.

| Assay | | | | | | Analysis | |
|---|---|---|---|---|---|---|---|
| Name | Primer 1 | Primer 2 | Probe 1 | Probe 2 | Tech. | Method | Gene |
| PM1901 | AAGGCAAAAG | TCCTTGTTCCGT | TACCTCGGC | NA | Real | REAL_TIME | ZmCENH3 |
| | GAGGGAACTG | CTTTTGCAG | GACGCC | | Time | | wild type |
| | AT (SEQ ID | (SEQ ID NO: | (SEQ ID NO: | | Assay | | allele |
| | NO: 98) | 99) | 100) | | | | |
| PM1909 | TCCTTGTTCCG | AAGGCAAAAGG | CCGGCGTCT | NA | Real | REAL_TIME | ZmCenH3 |
| | TCTTTTGCAG | AGGGAACTGAT | CGC (SEQ ID | | Time | | 10 bp |
| | (SEQ ID NO: | (SEQ ID NO: | NO: 103) | | Assay | | deletion |
| | 101) | 102) | | | | | |
| PM1913 | TCCTTGTTCCG | AAGGCAAAAGG | CGCGCTCAC | NA | Real | REAL_TIME | ZmCenH3 |
| | TCTTTTGCAG | AGGGAACTGAT | GCAC (SEQ | | Time | | 19 bp |
| | (SEQ ID NO: | (SEQ ID NO: | ID NO: 104) | | Assay | | deletion |
| | 101) | 102) | | | | | |
| SM0576CQ | CAGGTTCTTG | GATCACCGTCAA | TTTGATGCA | TCTTTGATG | TaqMan | END_POINT | C1 |
| | AGTCTCGGAA | CTTCAGAGTATG | GCTCACA | CTGCTCACA | | | (Zm00001d045065) |
| | TATTCA (SEQ | T (SEQ ID NO: | (SEQ ID NO: | (SEQ ID NO: | | | |
| | ID NO: 105) | 106) | 107) | 108) | | | |
| SM0956IQ | CAGAGATGGT | CAAAGGGTATC | TTGATGGC | TTTGATGGC | TaqMan | END_POINT | C1 |
| | GGTGGTGGAT | ACTGGAACAGG | ACGGGA | ATGGGAAG | | | (Zm00001d045065) |
| | GT (SEQ ID | AC (SEQ ID NO: | (SEQ ID NO: | (SEQ ID NO: | | | |
| | NO: 109) | 110) | 111) | 112) | | | |
| SM2669 | GCCCTCATGC | GACGACGAACA | ACGACCTTC | ACGACCTTT | TaqMan | END_POINT | Rf4 |
| | ACCTCATACC | TAAGAGGAGGA | GTAACG | GTAACGT | | | |
| | (SEQ ID NO: | TT (SEQ ID NO: | (SEQ ID NO: | (SEQ ID NO: | | | |
| | 113) | 114) | 115) | 116) | | | |
| SM2670 | GCCCTCATGC | CACCGTCGCCCT | AAGGTCGT | CGCGGACC | TaqMan | END_POINT | Rf4 |
| | ACCTCATACC | ATCAGTCT (SEQ | ACCAAATCC | AAATCC | | | |
| | (SEQ ID NO: | ID NO: 118) | (SEQ ID NO: | (SEQ ID NO: | | | |
| | 117) | | 119) | 120) | | | |
| SM2915 | TCAAGAGGGG | GTTTCCCCGCAA | CGCTCTTTT | CGCTCTTTA | TaqMan | END_POINT | CMS-C |
| | GGAGAACTAC | AAAGCTC (SEQ | AAAATAA | AAAATAA | | | |
| | C (SEQ ID | ID NO: 122) | (SEQ ID NO: | (SEQ ID NO: | | | |
| | NO: 121) | | 123) | 124) | | | |
| SM2916 | ACAAGATAGG | GAAATGCGCCC | TGAAGAAT | AAGAATGG | TaqMan | END_POINT | CMS-C |
| | GCCGTTCACA | GACCA (SEQ ID | GGATTTTAG | ATTTTCGCT | | | |
| | (SEQ ID NO: | NO: 126) | CTT (SEQ ID | TT (SEQ ID | | | |
| | 125) | | NO: 127) | NO: 128) | | | |
| SM6623 | CAAGTCACTG | CGAGGATCGAC | TACCATCGA | ACCATCGAT | TaqMan | END_POINT | R1 |
| | CTTCCGTCCAT | GCTTTGTTCA | CTTTTC | TTTTCA | | | (Zm00001d026147) |
| | T (SEQ ID | (SEQ ID NO: | (SEQ ID NO: | (SEQ ID NO: | | | |
| | NO: 129) | 130) | 131) | 132) | | | |
| SM8040 | CCCTGTTCCGA | CGTGAGCTTTCG | TCGATCGG | CGGGTGTG | TaqMan | END_POINT | C1 |
| | GGCCATTT | TCTGCGAT (SEQ | GTATGCTCT | CTCTCC | | | (Zm00001d045065) |
| | (SEQ ID NO: | ID NO: 134) | (SEQ ID NO: | (SEQ ID NO: | | | |
| | 133) | | 135) | 136) | | | |
| SM8091 | CTGCACGCCG | CAGCTGACGGC | CGACCGTA | ACCGTAACC | TaqMan | END_POINT | C1 |
| | CCAGATTA | AACAATTAGTA | ACCATGTAA | CTGTAACA | | | (Zm00001d045065) |
| | (SEQ ID NO: | (SEQ ID NO: | C (SEQ ID | (SEQ ID NO: | | | |
| | 137) | 138) | NO: 139) | 140) | | | |
| SM2918 | ATGGTGCCAA | ACCCCTCTGGTT | AATACCCTC | TATGAAGA | TaqMan | END_POINT | Mitochondrial |
| | TTCGTAATTTA | GCCTCTCT (SEQ | CAGTTTC | AGAATACC | | | genome |
| | AGTT (SEQ | ID NO: 142) | (SEQ ID NO: | ATCC (SEQ | | | |
| | ID NO: 141) | | 143) | ID NO: 144) | | | |
| SM4813 | GGCTAATATG | GGAGTATACAG | TTGAAACTG | CTTGAAACT | TaqMan | END_POINT | Mitochondrial |
| | GTGCCAATTC | ACCCCTCTGGTT | GAGGGTAT | GGATGGTA | | | genome |
| | GTA (SEQ ID | (SEQ ID NO: | TCT (SEQ ID | TTC (SEQ ID | | | |
| | NO: 145) | 146) | NO: 147) | NO: 148) | | | |
| SM2914 | GTATTCGCAC | AAAGCAAAAAT | CGTAAATTT | ATCGTAAAT | TaqMan | END_POINT | Mitochondrial |
| | CTACTCTGCCG | ACCATTGCAACC | TGTTTGATG | TTTTTTTGA | | | genome |
| | (SEQ ID NO: | (SEQ ID NO: | C (SEQ ID | TGC (SEQ ID | | | |
| | 149) | 150) | NO: 151) | NO: 152) | | | |
| SM4812 | GGCTTTATAG | TCGTAGCCACGT | CGGGAAGA | CCCGGGAC | TaqMan | END_POINT | Mitochondrial |
| | TCCTCAGAAA | GCTCTAATC | GATCCTGTG | CTGTGG | | | genome |
| | GGTGA (SEQ | (SEQ ID NO: | G (SEQ ID | (SEQ ID NO: | | | |
| | ID NO: 153) | 154) | NO: 155) | 156) | | | |
| SM0954BQ | CAGTGCGCCT | ACCAAGCCGGC | CATGTGCCT | CATGTGCCT | TaqMan | END_POINT | R1 |
| | CATACAGTTG | AAAGGAT (SEQ | AGGAGAG | CGGAGAG | | | (Zm00001d026147) |
| | TA (SEQ ID | ID NO: 158) | (SEQ ID NO: | (SEQ ID NO: | | | |
| | NO: 157) | | 159) | 160) | | | |
| SM6568 | CAAGTCACTG | GGATCGACGCTT | CACGACGA | CACGACGA | TaqMan | END_POINT | R1 |
| | CTTCCGTCCAT | TGTTCACCTG | AAGGATTA | AAGGATTT | | | (Zm00001d026147) |
| | T (SEQ ID | (SEQ ID NO: | AA (SEQ ID | AA (SEQ ID | | | |
| | NO: 161) | 162) | NO: 163) | NO: 164) | | | |
| SM0953BQ | GGCTTATAGC | CCTCTGCACCGC | TCAGCCTCA | CTCAGCCTT | TaqMan | END_POINT | R1 |
| | TTAGAGGCAC | CATCAA (SEQ ID | TCCTC (SEQ | ATCCTC | | | (Zm00001d026147) |
| | TTGAA (SEQ | NO: 166) | ID NO: 167) | (SEQ ID NO: | | | |
| | ID NO: 165) | | | 168) | | | |
| SM7200 | GTGGCCATTG | TGATGTGGTAGT | ATATGTACT | TGTACTTTC | TaqMan | END_POINT | Rf11 |
| | CATGTAAGC | TTCTGGATTGAA | TTCCCTCAC | CGTCACAA | | | |
| | (SEQ ID NO: | C (SEQ ID NO: | AAG (SEQ | G (SEQ ID | | | |
| | 169) | 170) | ID NO: 171) | NO: 172) | | | |
| SM5665 | GCAGACGTGA | TCCCTCGGAACA | TGTCGCTCG | CTGTCGCTG | TaqMan | END_POINT | Rf11 |
| | AGAGGATAGC | CAACAATGG | AAGCC | GAAGCC | | | |
| | A (SEQ ID | (SEQ ID NO: | (SEQ ID NO: | (SEQ ID NO: | | | |
| | NO: 173) | 174) | 175) | 176) | | | |

Step 4. We pollinated conversion line ears from the CHIP plants with pollen from the Recurrent Parent (DIP) plants and harvested the ears for embryo extraction at 16-19 days after pollination. The extracted embryos (i.e., the F1 generation) were placed on a petri dish containing 40 ml of Murashige and Skoog medium (MS) media (See generally Maluszynski, et al., eds., DOUBLED HAPLOID PRODUCTION IN CROP PLANTS: A MANUAL (2003). See also WO 2002/085104, incorporated herein by reference) with 0.5 mg/ml of colchicine or the same MS media without colchicine. The plates were placed in a Percival growth chamber at 28° C. under continuous light and 123 μmoles/m.see for 16-24 hours to allow embryos to express the color from the dominant R1-SCM2 allele.
Step 5. After 16 to 24 hours, white embryos (i.e., those lacking R1-SCM2 expression) were transferred to phytatrays containing 100 ml of germination medium and placed in a growth chamber with 16 hours of light, 118 μmoles/m.see at 28° C., and 8 hours of dark at 24° C. The germination media recipe contained MS salts, vitamins, and myo-inositol (See generally Murashige and Skoog, A Revised Medium for Rapid Growth and Bio Assays with Tobacco Tissue Cultures, Physiologia Plantarum 15:473-497 (1962)) with the addition of 0.5 ml/liter of Plant Preservative Mixture (PPM, Plant Cell Technology.)
Step 6. After about 10 days, we transplanted the surviving seedlings into 2.5-inch pots comprising soil and placed them in a Hardening chamber under the following environmental conditions: day temp−80° F., night temp−72° F., humidity-55%, photoperiod-12 hrs, light intensity-˜ 1200 μmols, CO2-400 ppm.
Step 7. Seedlings were sampled about 5 days after transplanting and genotyped with markers covering all 10 maize chromosomes. A subset of 144 plants that were found homozygous for all markers were selected for doubling (Table 13). We also confirmed the type of cytoplasm by testing them with two markers for CMS cytoplasm (SM2915 and SM2916).

TABLE 12
Number of F1 embryos extracted, seedlings
genotyped, and haploid seedlings.

| | Total embryos | | |
|---|---|---|---|
| | extracted | Seedlings | Haploid |
| Line | (colch + no.colch) | genotyped | seedlings |
| Line 1 | 510 | 256 | 66 |
| Line 2 | 767 | 277 | 61 |
| Line 3 | 676 | 314 | 27 |
| Line 4 | 391 | 44 | 25 |
| Line 5 | 231 | 42 | 11 |
| Line 6 | 146 | 10 | 6 |
| Line 7 | 841 | 423 | 42 |
| Line 8 | 439 | 163 | 9 |
| Line 9 | 148 | 43 | 15 |
| Line 10 | 1789 | 461 | 42 |
| Line 11 | 722 | 139 | 38 |
| Line 12 | 302 | 275 | 3 |
| Line 13 | 182 | 29 | 6 |

TABLE 13
Number of haploids selected for doubling per line
per treatment (colchicine v no colchicine).

| Line | Colchicine | No Colchicine | Total |
|---|---|---|---|
| Line 1 | 3 | 16 | 19 |
| Line 2 | 7 | 11 | 18 |
| Line 3 | 4 | 6 | 10 |
| Line 4 | 2 | 10 | 12 |
| Line 5 | 0 | 7 | 7 |
| Line 6 | 0 | 6 | 6 |
| Line 7 | 1 | 16 | 17 |
| Line 8 | 5 | 2 | 7 |
| Line 9 | 0 | 10 | 10 |
| Line 10 | 7 | 9 | 16 |
| Line 11 | 0 | 13 | 13 |
| Line12 | 0 | 3 | 3 |
| Line 13 | 0 | 6 | 6 |

Step 8. We calculated the haploid recovery rate (HRR) as the ratio of number of confirmed haploid seedlings per number of embryos extracted. Plants having either CENH3 mutation (10 bp deletion or 19 bp deletion) in a heterozygous state had a very similar HRR (Table 14).

TABLE 14
The average across all F1 families of rate of haploid
recovery for the two CENH3 mutations tested.

| CENH3 mutation | Average HRR | N |
|---|---|---|
| 509A115A (10 bp | 6.7 | 48 |
| deletion) | | |
| 509A151A (19 bp | 5.3 | 13 |
| deletion) | | |

All haploids were pollinated with pollen of Recurrent Parent plants to generate a HaploidBC1 generation.

TABLE 15
Double haploid seed produced by line and
treatment (Colchicine v No colchicine.)

| Line | Colchicine | No colchicine | Total |
|---|---|---|---|
| Line 1 | 449 | 134 | 583 |
| Line 2 | 33 | 2 | 35 |
| Line 3 | 411 | 46 | 457 |
| Line 4 | 10 | 79 | 89 |
| Line 5 | NA | 6 | 6 |
| Line 6 | NA | 17 | 17 |
| Line 7 | 2 | 45 | 47 |
| Line 8 | 192 | 12 | 204 |
| Line 9 | NA | 15 | 15 |
| Line 10 | 33 | 3 | 36 |
| Line 11 | NA | 0 | 0 |
| Line 12 | NA | 5 | 5 |
| Line 13 | NA | 1 | 1 |

After pollinations, the ears were left to dry down. Another round of embryo rescue and pollinations by Recurrent Parent will be completed to bulk up the DH seed.

### Example 5. General Method to Convert Non-CMS Recurrent Parent Lines to CMS Lines

Step 1. DIPs are selected to be converted to CMS. Selected lines need to be homozygous (ideally) or heterozygous for rf4 (the recessive allele that confers male sterility when combined with CMS cytoplasm). These lines are rf4 recurrent parents.
Step 2. The CHIP is grown, and individual plants are genotyped for CenH3, as well as for markers for the CMS, Rf4 and R1 loci, if necessary. Plants heterozygous for the CenH3 knockout allele are used for CMS cyto-swapping. In any inducer population, there will be many plants that are homozygous WT for the CenH3 gene. These plants are not inducers and must be sorted away. Selected CHIP plants are optionally R1-SCM2 or R1-nj homozygous. In the ideal one-step cyto-swapping method, one of these two alleles are already fixed in the line. The inducer line may be increased by self-pollination (if they are male fertile) or through crossing by a maintainer line's pollen (if they are male sterile). The maintainer line would have the R1-SCM2 or R1-nj color marker to keep that fixed in the inducer line. The maintainer could have the non-restorer alleles of rf4 and rf11 as well as CenH3 WT or CenH3 knockout (mutant) alleles.
Step 3. The DIPs are crossed as males (pollen donors) onto the CHIPs.
Step 4. If the R1-nj marker is used, the resulting seed is grown to maturity, dried and harvested, and sorted for haploids (cream-colored embryos), which are then planted. In contrast, if the R1-SCM2 marker is used, the resulting ears are harvested between ten and twenty-five days after pollination. Embryos from the kernels are isolated and incubated in appropriate media (referred to as embryo rescue media) suitable for maintaining the embryos' viability. In one embodiment, the rescue media used for haploid induction rate (HIR) determination comprises 4.43 grams of Murashige and Skoog basal media with vitamins, 30 grams of sucrose, and 70 mg of salicylic acid. The embryos in the rescue media are placed under conditions to allow the expression of the color indicator gene (e.g., R1-SCM2). In exemplary embodiments, the embryos are placed under 100-400 micromol light for 16-24 hours at 22-31° C. until some of the embryos turn purple due to the expression of the R1-SCM2 gene (see protocol, for example, described in WO2015/104358). The purple (diploid) and cream-colored (haploid) embryos can be counted from each ear. The frequency of haploids, known as the HIR or haploid induction rate, can be determined based on the number of haploids over the total embryos. Optionally, a colchicine treatment is applied to induce genome doubling at some point during this process. See generally Maluszynski, et al., eds., DOUBLED HAPLOID PRODUCTION IN CROP PLANTS: A MANUAL (2003). See also WO 2002/085104, incorporated herein by reference. In one embodiment, the colchicine is co-applied in the rescue media described above.
Step 5. The DIP seed is planted so it will nick (its pollen shedding occurs simultaneously with ears being receptive-silking-on the progeny CMS-converted haploid plants) and be used as a pollen donor for the haploid plants when they flower. However, optionally, one could also simply use stored or preserved pollen here as a donor for the flowering haploid plants.
Step 6. Haploid plantlets are sampled and genotyped. Plants carrying the markers for the CMS cytoplasm and paternal genotypes for the other assays are confirmed as paternal haploids. At the very least, the haploids are genotyped for the CenH3 gene, and the haploids contain the wild type (non-edited) allele. If tested, the haploids will have the rf4 allele, and any other allele from the DIP (paternal) genome. Regardless of whether they were treated with a doubling agent, the haploids are expected to be male sterile because of the CMS cytoplasm and rf4 allele. In other words, through genotyping, the putative haploid plants (sorted by embryo color) may optionally be confirmed as cyto-swapped haploids by genotyping if the genotyping result is that they do not carry an edited CenH3 allele, and that they have the CMS cytoplasm genotype markers and all other nuclear markers come out as DIP parent (rather than CHIP) calls.
Step 7. The haploid plants are pollinated with DIP parent pollen. If the haploids are not treated with a chemical doubling agent, then any seed set (implied female fertility) will be a result of the natural biological process of spontaneous doubling in the female inflorescence (ear), which is known to be common in maize germplasm. Treatment of the embryos with a chemical doubling agent may improve the seed set of the ear by generating doubled haploid sectors. Overall, without wishing to be bound by theory, it is expected that the CMS cyto-swapping pipeline may be run with or without a doubling step with nearly any maize germplasm due to the fact that the haploid ear will have some ovules or embryo sacs that spontaneously double and those may be fertilized by recurrent parent pollen, e.g., a backcross, to set pure “HaploidBC1” seed. HaploidBC1 is a cross between the recurrent parent and the haploid genome, and if there is any variation in the parental line (i.e., if the recurrent parent is not a fixed inbred) then that variation may be apparent in different cyto-swapped lines coming out of the process.
Step 8. After maturation and dry down, the haploid plant's pollinated ear(s) are harvested, and the resulting seed is planted alongside the DIP parent (which will act as a pollen donor again). Crosses are made once again to bulk up the seed of the CMS line. In this generation, the CMS line may be genotyped again to confirm the status and purity of the conversion and to verify the absence of the CHIP nuclear DNA (including the Rf4 and CenH3 mutant markers).
Step 9. When desiring to make hybrid seed, the CMS cyto-swapped line may be planted as the female alongside a male line. The female CMS line should be male sterile so it can be crossed easily by the pollen donor in the hybrid production field without significant human intervention.

TABLE 16
Construct Annotations. Construct 26258:

| Name | Type | Start | End | Length | Direction |
|---|---|---|---|---|---|
| oCOLE-06 | rep_origin | 16644 | 17450 | 807 | reverse |
| oVS1-03 | rep_origin | 15562 | 15966 | 405 | forward |
| cRepA-05 | CDS | 14446 | 15519 | 1074 | forward |
| cVirG-06 | CDS | 13691 | 14416 | 726 | forward |
| cSpec-03 | CDS | 12603 | 13391 | 789 | forward |
| bNLB-05 | insertion_seq | 12194 | 12323 | 130 | reverse |
| tUbi1-06 | terminator | 11077 | 12111 | 1035 | forward |
| cPMI-14 | CDS | 9889 | 11064 | 1176 | forward |
| prUbi1-18 | promoter | 7889 | 9881 | 1993 | forward |
| ZmUPL3\target | misc_feature | 7852 | 7873 | 22 | forward |
| rLbgRNACas12aZmUPL3-01 | misc_RNA | 7831 | 7873 | 43 | forward |
| rLbCrRNA-01 | misc_RNA | 7831 | 7851 | 21 | forward |
| ZmUBL\target | misc_feature | 7809 | 7830 | 22 | forward |
| rLbgRNACas12aZmUBL-01 | misc_RNA | 7788 | 7830 | 43 | forward |
| rLbCrRNA-01 | misc_RNA | 7788 | 7808 | 21 | forward |
| prOsU6-01 | promoter | 7271 | 7786 | 516 | forward |
| ZmWaxy1\target | misc_feature | 7233 | 7255 | 23 | forward |
| rLbgRNACas12aZmWaxy1- | misc_RNA | 7212 | 7255 | 44 | forward |
| 01 | | | | | |
| rLbCrRNA-01 | misc_RNA | 7212 | 7232 | 21 | forward |
| ZmYellow1\target | misc_feature | 7189 | 7211 | 23 | forward |
| rLbgRNACas12aZmYellow1- | misc_RNA | 7168 | 7211 | 44 | forward |
| 01 | | | | | |
| rLbCrRNA-01 | misc_RNA | 7168 | 7188 | 21 | forward |
| ZmOpaque2\target | misc_feature | 7145 | 7167 | 23 | forward |
| rLbgRNACas12aZmO2-01 | misc_RNA | 7124 | 7167 | 44 | forward |
| rLbCrRNA-01 | misc_RNA | 7124 | 7144 | 21 | forward |
| prOsU6-01 | promoter | 6607 | 7122 | 516 | forward |
| tNOS-05-01 | terminator | 6327 | 6579 | 253 | forward |
| cLbCas12a-27 | CDS | 2037 | 6318 | 4282 | forward |
| prSoUbi4-04 | promoter | 222 | 2018 | 1797 | forward |
| bNRB-01-01 | insertion_seq | 101 | 125 | 25 | reverse |

1. A method for conferring cytoplasmic male sterility (CMS) on a plant line, the method comprising
a. obtaining a first plant comprising a CMS cytoplasm, wherein the first plant is a haploid inducer (CHIP);
b. obtaining a second plant comprising a desired nuclear genome (DIP); and
c. crossing the first plant with the second plant; and
d. generating a progeny therefrom;
i, wherein the progeny comprises the CMS cytoplasm and the desired nuclear genome.

2. The method of claim 1, wherein the CMS cytoplasm is selected from the group consisting of CMS-C, CMS-S, and CMS-T.

3. (canceled)

4. The method of claim 1, wherein the CHIP is female fertile and CMS male fertile or female fertile and CMS male sterile.

5. (canceled)

6. The method of claim 4, wherein the CHIP is a paternal haploid inducer.

7. The method of claim 6, wherein the CHIP comprises a cenh3 mutation.

8. The method of claim 7, wherein the cenh3 mutation is a heterozygous knockout mutation obtained by gene editing.

9. (canceled)

10. The method of claim 8, wherein the cenh3 knockout mutation comprises SEQ ID NO: 5 or SEQ ID NO: 6.

11. (canceled)

12. The method of claim 8, wherein the cenh3 knockout mutation is edited using CRISPR-Cas 12a, wherein the CRISPR-Cas 12a is selected from the group consisting of AsCas12a, LbCas12a, and FnCas12a, MbCas12a, Mb2Cas12a, etc.

13. (canceled)

14. (canceled)

15. The method of claim 7, wherein the CHIP further comprises a homozygous anthocyanin marker, wherein the anthocyanin marker is selected from the group consisting of R1-navajo and R1-SCM2.

16. (canceled)

17. (canceled)

18. (canceled)

19. (canceled)

20. The method of claim 7, wherein the CHIP further comprises a homozygous restorer allele selected from the group consisting of Rf3, Rf4, Rf10, Rf11, and Rf12.

21. (canceled)

22. The method of claim 4, wherein the CHIP comprises a homozygous non-restorer allele, wherein the non-restorer allele is selected from the group consisting of rf3, rf4, rf10, rf11, and rf12.

23. (canceled)

24. (canceled)

25. (canceled)

26. (canceled)

27. (canceled)

28. (canceled)

29. (canceled)

30. (canceled)

31. The method of claim 1, wherein the CHIP is selected from the group consisting of maize, wheat, rice, sunflower, tomato, barley, brassicas, cucumber, and watermelon.

32. (canceled)

33. The method of claim 1, wherein the DIP is a pollen donor in the cross of step c.

34. The DIP of claim 1, wherein the DIP is homozygous or heterozygous for a non-restorer allele.

35. The DIP of claim 34, wherein the DIP is homozygous for the non-restorer allele, wherein the non-restorer allele is selected from the group consisting of rf3, rf4, rf10, rf11, and rf12.

36. (canceled)

37. (canceled)

38. The method of claim 1, wherein the DIP is selected from the group consisting of maize, wheat, rice, and sunflower, tomato, barley, brassicas, cucumber, and watermelon.

39. (canceled)

40. A plant produced by the method of claim 1, wherein the plant is a CMS haploid plant.

41. The CMS haploid plant of claim 40, wherein the CMS haploid plant comprises the CMS cytoplasm of the CHIP and the nuclear genome of the DIP.

42. The CMS haploid plant of claim 41, wherein the CMS haploid plant lacks an anthocyanin marker, a restorer allele, and a cenh3 knockout mutation.

43. The CMS haploid plant of claim 42, wherein the CMS haploid plant is treated with a doubling agent selected from the group consisting of colchicine, pronamide, dithipyr, trifluralin, nitrous oxide, or another known anti-microtubule agent.

44. (canceled)

45. (canceled)

46. The CMS haploid plant of claim 42, wherein the CMS haploid plant is pollinated with pollen from the DIP or with preserved pollen.

47. (canceled)

48. The CMS haploid plant of claim 40, wherein the CMS haploid plant is confirmed CMS by genotyping or other molecular analysis.


SEQUENCE LISTING (177 sequences)

SEQ ID NO: 1 - DNA, 23 bp, synthetic construct
  source 1..23: mol_type = other DNA
caggtggtgcgagtacctcggcg

SEQ ID NO: 2 - DNA, 21 bp, synthetic construct
  source 1..21: mol_type = other DNA
tccttgttccgtcttttgcag

SEQ ID NO: 3 - DNA, 22 bp, synthetic construct
  source 1..22: mol_type = other DNA
aaggcaaaaggagggaactgat

SEQ ID NO: 4 - DNA, 15 bp, synthetic construct
  source 1..15: mol_type = other DNA
tacctcggcgacgcc

SEQ ID NO: 5 - DNA, 19 bp, synthetic construct
  source 1..19: mol_type = other DNA
tacctcggcgacgccggtg

SEQ ID NO: 6 - DNA, 10 bp, synthetic construct
  source 1..10: mol_type = other DNA
gtacctcggc

SEQ ID NO: 7 - DNA, 191 bp, Zea mays
  source 1..191: mol_type = genomic DNA
ctctccgaccctggtgctaagcacgttccttgttccgtcttttgcaggtggtgcgagtacctcggcgacgccggtgagcgcgtgcgtgcggggatcagttccctccttttgcctttttttgttgggctgctcttacttgcttgcaagctgtttgatggaatgcaggaaagggctgctgggaccgggggaag

SEQ ID NO: 8 - DNA, 179 bp, Zea mays
  source 1..179: mol_type = genomic DNA
ctctccgaccctggtgctaagcacgttccttgttccgtcttttgcaggtggtgcgagacgccggtgagcgcgtgcgtgcggggatcagttccctccttttgccttttttgtgggctgctcttacttgcttgcaagctgtttgatggaatgcaggaaagggctgctgggaccgggggaag

SEQ ID NO: 9 - DNA, 170 bp, Zea mays
  source 1..170: mol_type = genomic DNA
ctctccgaccctggtgctaagcacgttccttgttccgtcttttgcaggtggtgcgtgagcgcgtgcgtgcggggatcagttccctccttttgccttttttgtgggctgctcttacttgcttgcaagctgtttgatggaatgcaggaaagggctgctgggaccgggggaag

SEQ ID NO: 10 - protein, 115 aa, Zea mays
MARTKHQAVRKTAEKPKKKLQFERSGGASTSATPERAAGTGGRAASGGDSVKKTKPRHRWRPGTVALREIRKYQKSTEPLIPFAPFVRVVRELTNFVTNGKVERYTAEALLALQE

SEQ ID NO: 11 - protein, 109 aa, Zea mays
MARTKHQAVRKTAEKPKKKLQFERSGGASTSATPERAAGTGGRAASGGDSVKKTKPRHRWRPGLRCGRSGSTRSPLNRSGSPISQTGKSAIPQKPSLRCKSPLRLSSVW

SEQ ID NO: 12 - protein, 106 aa, Zea mays
MARTKHQAVRKTAEKPKKKLQFERSGGARRRKGLLGPGEERRLEVTQLRRRNHATAGGQGLRCGRSGSTRSPLNRSSPLRLSSVWGSPISQTGKSAIPQKPSLRCK

SEQ ID NO: 13 - protein, 112 aa, Zea mays
MARTKHQAVRKTAEKPKKKLQFERSGGASTSATPVSAKGLLGPGEERRLEVTQLRRRNHATAGGQGLRCGRSGSTRSPLNRSSPLRLSSVWGSPISQTGKSAIPQKPSLRCK

SEQ ID NO: 14 - protein, 119 aa, Zea mays
MARTKHQAVRKTAEKPKKKLQFERSGGASTSATPVSACERAAGTGGRAASGGDSVKKTKPRHRWRPGTVALREIRKYQKSTEPLIPFAPFVRVVRELTNFVTNGKVERYTAEALLALQE

SEQ ID NO: 15 - protein, 123 aa, Zea mays
MARTKHQAVRKTAEKPKKKLQFERSGGASTSATPVSACVRGSERAAGTGGRAASGGDSVKKTKPRHRWRPGTVALREIRKYQKSTEPLIPFAPFVRVVRELTNFVTNGKVERYTAEALLALQE

SEQ ID NO: 16 - protein, 125 aa, Zea mays
MARTKHQAVRKTAEKPKKKLQFERSGGASTSATPVSACVRGSVPSFCLFLKGLLGPGEERRLEVTQLRRRNHATAGGQGLRCGRSGSTRSPLNRSSPLRLSSVWGSPISQTGKSAIPQKPSLRCK

SEQ ID NO: 17 - protein, 136 aa, Zea mays
MARTKHQAVRKTAEKPKKKLQFERSGGASTSATPVSACVRGSVPSFCLFLLGCSYLLASKGCWDRGKSGVWRLSEDETTPPLAARDCSAAGDQEVPEVHTAHPLCAFRPCGEGVNQFRNKRESRALYRRSPPCAAR

SEQ ID NO: 18 - protein, 112 aa, Zea mays; identical to SEQ ID NO: 13

SEQ ID NO: 19 - protein, 119 aa, Zea mays; identical to SEQ ID NO: 14

SEQ ID NO: 20 - protein, 123 aa, Zea mays; identical to SEQ ID NO: 15

SEQ ID NO: 21 - protein, 125 aa, Zea mays; identical to SEQ ID NO: 16

SEQ ID NO: 22 - protein, 136 aa, Zea mays; identical to SEQ ID NO: 17

SEQ ID NO: 23 - DNA, 23 bp, synthetic construct
  source 1..23: mol_type = other DNA
gctactataccatagcaatagac

SEQ ID NO: 24 - DNA, 18 bp, synthetic construct
  source 1..18: mol_type = other DNA
ctatacggcagctcgata

SEQ ID NO: 25 - DNA, 18 bp, synthetic construct
  source 1..18: mol_type = other DNA
aatgatgcactaataaac

SEQ ID NO: 26 - DNA, 16 bp, synthetic construct
  source 1..16: mol_type = other DNA
tgatgcactactaaac

SEQ ID NO: 27 - DNA, 303 bp, Zea mays
  source 1..303: mol_type = genomic DNA
  variation 152: note = m = a or c
cagggccatcgacaccagctgcaccagtgtttagtccacctaaagcaaggaaggggatcaacataaggcatcagctgtacacatgaaagattgaaagagtgctactataccatagcaatagacctagaattgaagtgttaatgatgcactamtaaactggagaacaaatgaatattcagaataaaaattgacaaataaataaacagaagaatactcccaatcttcattcagacatatcgagctgccgtatagccagattggtacacacgatttatccttctttttgctcgggcgcaaggtaaa

SEQ ID NO: 28 - DNA, 26 bp, synthetic construct
  source 1..26: mol_type = other DNA
gtgatcgctctgaatgtttaatgtgt

SEQ ID NO: 29 - DNA, 25 bp, synthetic construct
  source 1..25: mol_type = other DNA
ttcaacacctcagtacatcgctaaa

SEQ ID NO: 30 - DNA, 14 bp, synthetic construct
  source 1..14: mol_type = other DNA
catgcgcgtgtcct

SEQ ID NO: 31 - DNA, 17 bp, synthetic construct
  source 1..17: mol_type = other DNA
aatcatgcgcctgtcct

SEQ ID NO: 32 - DNA, 499 bp, Zea mays
  source 1..499: mol_type = genomic DNA
  variation 69: note = s=c or g
  variation 278: note = n= a or c or g or t/u
gaaggcgtggcatcagctatgaaactatctgtgttcaacacctcagtacatcgctaaaatacaggacasgcgcatgatttcccacacattaaacattcagagcgatcacctgaaccgggctatccttgtcaacaacaaacgatctagctaccttgcctgaaggcgtgttcacttccactgtgtacgaaccatggtatcctctgaacttaaactctccttggtgatcgacactgccgttcatacgggtcagccactcttgtttgagagcaaggtacctnctgccagcctcgttgattgtcccatcagcatcgaccagatgggcgtgctctcgaaacataaacatctcccagaatccccagaggatgattccccccacagcaggatgtgcaaatgcttcccggaggaacacctctagatcatcagctcgtatgtgctcgttctctgctgtgacatccaactcagtaatccagatcgggagacccagtatggcgagcttgtc

SEQ ID NO: 33 - DNA, 31 bp, synthetic construct
  source 1..31: mol_type = other DNA
ctctgctattttagattgcttaggattcagt

SEQ ID NO: 34 - DNA, 25 bp, synthetic construct
  source 1..25: mol_type = other DNA
gacgaattgcaaaagtacgaagcta

SEQ ID NO: 35 - DNA, 19 bp, synthetic construct
  source 1..19: mol_type = other DNA
cttagatgtacaaattaag

SEQ ID NO: 36 - DNA, 18 bp, synthetic construct
  source 1..18: mol_type = other DNA
ttagatgtacagattaag

SEQ ID NO: 37 - DNA, 295 bp, Zea mays
  source 1..295: mol_type = genomic DNA
  variation 17: note = y = c or t/u
  variation 35: note = r = a or g
  variation 38: note = m = a or c
  variation 145: note = r = a or g
  variation 175: note = r = a or g
  variation 188: note = v = a or c or g, not t/u
  variation 199: note = m = a or c
  variation 238: note = s = c or g
cttctacgtccttctaycttttgtgtgtgttttgrgtmctacttttgcaaagtaaatgtctgaaatagcaaatgttcctaattttgcaaagtaaatgtgacgaattgcaaaagtacgaagctagcttagtcgcttagatgtacarattaaggtagatcgccaccaaaataaacarcaccatgcagcavaccacaaaccmactgaatcctaagcaatctaaaatagcagagttcaaaasattcaactaagccccgcacgcagaactaccagaacccatcattgccaaatctgagag

SEQ ID NO: 38 - DNA, 24 bp, synthetic construct
  source 1..24: mol_type = other DNA
ccttggtggtggtatgatctacac

SEQ ID NO: 39 - DNA, 24 bp, synthetic construct
  source 1..24: mol_type = other DNA
ttccacgagagattttccaacaga

SEQ ID NO: 40 - DNA, 17 bp, synthetic construct
  source 1..17: mol_type = other DNA
ttacaaggcacagggat

SEQ ID NO: 41 - DNA, 18 bp, synthetic construct
  source 1..18: mol_type = other DNA
ttacaaggcacaaggata

SEQ ID NO: 42 - DNA, 624 bp, Zea mays
  source 1..624: mol_type = genomic DNA
  variation 10: note = n = a or c or g or t/u
  variation 16: note = n = a or c or g or t/u
  variation 22: note = n = a or c or g or t/u
  variation 36: note = n = a or c or g or t/u
  variation 40: note = n = a or c or g or t/u
  variation 121: note = n = a or c or g or t/u
  variation 130: note = n = a or c or g or t/u
  variation 223: note = n = a or c or g or t/u
  variation 279: note = n = a or c or g or t/u
  variation 294: note = n = a or c or g or t/u
  variation 318: note = n = a or c or g or t/u
  variation 386: note = n = a or c or g or t/u
  variation 466: note = n = a or c or g or t/u
  variation 492: note = n = a or c or g or t/u
  variation 514: note = n = a or c or g or t/u
  variation 580: note = r = a or g
  variation 623: note = n = a or c or g or t/u
cattggtgangaagtngagaanttggctgccgcttngccngaaggtggagttctgctcctagagaatgttagattctacaaggaggaagagaagaacgaacctgagtttgctaagaagctngcatctgtngctgacctttatgtcaatgatgcttttggcacggcacacagagctcatgcttcaaccgaaggagttaccaagtatttgaagcctgctgttgcnggcttcctcatgcagaaggtaataaaatgtcacccatgctcatgatcttttaagtnatttaggtactgttnagtaggatggggtaaatatttttntacctttcaaaagagaccaataaatctttttaacatttcagttttttcataaataaaaaaaggaaagntttgacaaatgactttattatctcaggaacttgactaccttgttggagctgttgccaacccaaagaagccatttgccgcnattgttggtggatccaaggtctcaantaagattggtgtcattgagtcnttgctggcgaaggtcgatatcctcatccttggtggtggtatgatctacacattttacaaggcacarggatattctgttggaaaatctctcgtggaaagaggataaactng

SEQ ID NO: 43 - DNA, 25 bp, synthetic construct
  source 1..25: mol_type = other DNA
ttggctttaaatagcttatgagcac

SEQ ID NO: 44 - DNA, 22 bp, synthetic construct
  source 1..22: mol_type = other DNA
cgacgaatgcatgatccagaac

SEQ ID NO: 45 - DNA, 20 bp, synthetic construct
  source 1..20: mol_type = other DNA
ctggcaatttggtaaatata

SEQ ID NO: 46 - DNA, 19 bp, synthetic construct
  source 1..19: mol_type = other DNA
ctggcaatttggtaaacat

SEQ ID NO: 47 - DNA, 96 bp, Zea mays
  source 1..96: mol_type = genomic DNA
  variation 46: note = r = a or g
tcgacgaatgcatgatccagaactggttatctttcggataaatatrtttaccaaattgccagctagtgctcataagctatttaaagccaaattgcc

SEQ ID NO: 48 - DNA, 24 bp, synthetic construct
  source 1..24: mol_type = other DNA
acaaatccacagtagtaggcgagt

SEQ ID NO: 49 - DNA, 23 bp, synthetic construct
  source 1..23: mol_type = other DNA
gccacagatgcgagtgaaatcag

SEQ ID NO: 50 - DNA, 16 bp, synthetic construct
  source 1..16: mol_type = other DNA
ccatgatgcgtgatca

SEQ ID NO: 51 - DNA, 16 bp, synthetic construct
  source 1..16: mol_type = other DNA
catgatgcgtgaccat

SEQ ID NO: 52 - DNA, 205 bp, Zea mays
  source 1..205: mol_type = genomic DNA
  variation 140: note = y = c or t/u
  variation 151: note = r = a or g
  variation 190: note = y = c or t/u
gcaagatgctttaygcaagctcaaaccagaaattcaagagtggcctcaatggcattcaggttgaactccaggccacagatgcgagtgaaatcagcctcgatgagatcaaggatcgggcacgctaggcgcaccgccatcgygagtttcatgrtcacgcatcatggactcgcctactactgtggatttgtaygccattatagacttg

SEQ ID NO: 53 - DNA, 23 bp, synthetic construct
  source 1..23: mol_type = other DNA
cctcaactttgatcttcttcgca

SEQ ID NO: 54 - DNA, 17 bp, synthetic construct
  source 1..17: mol_type = other DNA
tgctcctgagccagaga

SEQ ID NO: 55 - DNA, 17 bp, synthetic construct
  source 1..17: mol_type = other DNA
cagaagcatcagtatcc

SEQ ID NO: 56 - DNA, 16 bp, synthetic construct
  source 1..16: mol_type = other DNA
agcatcagtaccctca

SEQ ID NO: 57 - DNA, 279 bp, Zea mays
  source 1..279: mol_type = genomic DNA
  variation 140: note = r = a or g
cttcccactggtggtgatgtttcaattgccagcattgcagctgctcctgagccagagaaggtgaaggtggagccggacacagctgtgactgaagaggtcaaggagaagaaaaaaaagaggcagaaggatgatgctgaggrtactgatgcttctgttcctgcgaagaagatcaaagttgaggaagccactgaagcagtggaaggggagaagagtgagaagaagaagaagaaaaagaaagacaagggcgagtcagagccagctgaggtcgttgagggcaag

SEQ ID NO: 58 - DNA, 23 bp, synthetic construct
  source 1..23: mol_type = other DNA
gagttagtttctttgggtgcaga

SEQ ID NO: 59 - DNA, 21 bp, synthetic construct
  source 1..21: mol_type = other DNA
aacatagggcgatcatcagtc

SEQ ID NO: 60 - DNA, 16 bp, synthetic construct
  source 1..16: mol_type = other DNA
ttctgtgaagctgaca

SEQ ID NO: 61 - DNA, 16 bp, synthetic construct
  source 1..16: mol_type = other DNA
agttctgtgaagccga

SEQ ID NO: 62 - DNA, 227 bp, Zea mays
  source 1..227: mol_type = genomic DNA
  variation 62: note = w = a or t/u
  variation 79: note = r = a or g
  variation 179: note = r = a or g
  variation 189: note = w= a or t/u
tcaaggggctaacatagggcgatcatcagtcttgcaaaatacagagaacaaccttaaaactwcttcaggaagactgtcrgcttcacagaactcagattctgcacccaaagaaactaactcggcaccttgtatgttcttgctcatctctatctactcatttccacatttaatgrttaaagcatttcatawgccttatctgtcttttatctgttcttaatactgaagca

SEQ ID NO: 63 - DNA, 16 bp, synthetic construct
  source 1..16: mol_type = other DNA
aagctcgacaagaagg

SEQ ID NO: 64 - DNA, 20 bp, synthetic construct
  source 1..20: mol_type = other DNA
gactgggcgttccttgatca

SEQ ID NO: 65 - DNA, 16 bp, synthetic construct
  source 1..16: mol_type = other DNA
aagctcgaccagaagg

SEQ ID NO: 66 - DNA, 16 bp, synthetic construct
  source 1..16: mol_type = other DNA
aagctcgacaagaagg

SEQ ID NO: 67 - DNA, 101 bp, Zea mays
  source 1..101: mol_type = genomic DNA
  variation 51: note = m = a or c
atgttgatttttctgacagcgcgtctcttgctgctgctttgaagctcgacmagaaggtgatcaaggaacgcccagtcagaatcagatgtgctgttccaaaa

SEQ ID NO: 68 - DNA, 17 bp, synthetic construct
  source 1..17: mol_type = other DNA
gcgccaacactgtctga

SEQ ID NO: 69 - DNA, 23 bp, synthetic construct
  source 1..23: mol_type = other DNA
gtcgtaacgtttgcacatccaat

SEQ ID NO: 70 - DNA, 19 bp, synthetic construct
  source 1..19: mol_type = other DNA
tctattgatctggactgtg

SEQ ID NO: 71 - DNA, 18 bp, synthetic construct
  source 1..18: mol_type = other DNA
tctattgatccggactgt

SEQ ID NO: 72 - DNA, 101 bp, Zea mays
  source 1..101: mol_type = genomic DNA
  variation 51: note = r = a or g
cgtcgcgcgccaacactgtctgagctgagctgtgattgtcgccacagtccrgatcaatagatcattggatgtgcaaacgttacgacaatgatacaagggtc

SEQ ID NO: 73 - DNA, 24 bp, synthetic construct
  source 1..24: mol_type = other DNA
tgttgcattgcattccagttgttg

SEQ ID NO: 74 - DNA, 20 bp, synthetic construct
  source 1..20: mol_type = other DNA
tcgctcgtcgtctctgtcat

SEQ ID NO: 75 - DNA, 15 bp, synthetic construct
  source 1..15: mol_type = other DNA
catcgcgggatgttc

SEQ ID NO: 76 - DNA, 15 bp, synthetic construct
  source 1..15: mol_type = other DNA
tacatcgccggatgt

SEQ ID NO: 77 - DNA, 801 bp, Zea mays
  source 1..801: mol_type = genomic DNA
  variation 55: note = y = c or t/u
  variation 263: note = r = a or g
  variation 307..316: note = n = a or c or g or t/u
  variation 356: note = n = a or c or g or t/u
  variation 333: note = y = c or t/u
  variation 368: note = k = g or t/u
  variation 370: note = r = a or g
  variation 377: note = y = c or t/u
  variation 401: note = s = c or g
  variation 451: note = k = g or t/u
  variation 461: note = y = c or t/u
  variation 571..574: note = n = a or c or g or t/u
  variation 619: note = w = a or t/u
  variation 640: note = y = c or t/u
  variation 736: note = n = a or c or g or t/u
  variation 752..758: note = n = a or c or g or t/u
ccggtaccacgagcagctgctccgcctggtgcgcgtcgggggtaccgtcgtgtaygacaacacgctgtgggcsggtactgtggcgcttccccccgacgcgccgctcagcgacctcgaccgcaggttctccgccgccatcagggaactcaacgtccggctttctcaggatccccgcgtcgaggtctgccagctcgccatcgccgacggcgtcaccatctgccgccgcgtcgtctgatgtgatgatgatccgacgaccaagatcrtatatcattcgctcgtcgtctctgtcatctttcaactgcctgcnnnnnnnnnnccgctgccgtcgtcaaytaataatgcatggttcttgttcntttttttttttktrcttgcaytgtgtgtgttgagttgaacatccsgcgatgtactgcaacaactggaatgcaatgcaacatcatgcgtgcagctktaactaaaaytgacgacgacagcgtcgtccatgcatggaccgaggcggttatattaggtgtcgatcatccatgagtgaccgaggcggttaggtgtcgatccatgagtgacggagagtggnnnncggccggcggcgaggccatgattggcgcgtcgtcgggtaggggawgggatgggcttgcactgtagyctgcggggagataagcctcctcggtcggtcgggatggcgggcgtgtcgttgtcrtctcgtccgccagcccggcacggagggcaggaggtggaagcnaagaatcacagtcgtnnnnnnncgtaccctacccatggagacatgcatgcatatcctcagagaga

SEQ ID NO: 78 - DNA, 24 bp, synthetic construct
  source 1..24: mol_type = other DNA
caatccattcctgtgattacgcat

SEQ ID NO: 79 - DNA, 20 bp, synthetic construct
  source 1..20: mol_type = other DNA
gctgttactcgctctgctac

SEQ ID NO: 80 - DNA, 19 bp, synthetic construct
  source 1..19: mol_type = other DNA
tgccaatgtgataaatgaa

SEQ ID NO: 81 - DNA, 19 bp, synthetic construct
  source 1..19: mol_type = other DNA
tgccaatgtgatacatgaa

SEQ ID NO: 82 - DNA, 1001 bp, Zea mays
  source 1..1001: mol_type = genomic DNA
  variation 501: note = m = a or c
gtgagtggaaagttgaattccaattccacgtacgccaaacaggtttggccaggtatcaaattctaattcagcccaattccgactaaacaaacaggcccttatggcaaagtacttgcttggattcaagaatagggctgctccatacatcttttgctccatttggttttcccatctcttctcatagtattctagtacttcttttaaatgaacttgatttgtctttcaaagaatccttaatgacagtcttcgtaacatccatggctgccataatctcaagagctgcttgtgcctaatttccatctgctatccttagagcatcaagaagtggttgtgaagctcttaggcaatattccactttattccaaaatggcattgaaacaacaatgttctcaacttgccggcctttatgagaggtagataagctggtagcatgccattcatcactaacgaataaatttctcaatccattcctgtgattacgcatgcttgccaatgtgatamatgaagtagcagagcgagtaacagctgacttcacaagatctccacctttttttgtcgctcattagatcaagaaccttcccatgcttgtagacgaatccacatacctttgccttgttgatgagtgttgaattctttgatcttcccaatgtccaacatcacatctaagcaatgagcagtacaaggtgtcttgtgtctaaatgttggatcctctccattagaagcatctctgctgccttgaagttggctccattgtcagtgacaatctgcaccacatgttccttcccaaccttatcaatttgcttctccaacatgtcacccaacatattttcattttctgtctcacttgaagcatcaacagactctaggaagaatgtccctgctggaatgttagcaaggaaattgataagatggtggtggcttgtgtcagtccattcatcagatattatggtgcagccatattccttccaagcctcctcatgcttattctcatcactgatgtt

SEQ ID NO: 83 - DNA, 20 bp, synthetic construct
  source 1..20: mol_type = other DNA
ctcgctctaccaggccatta

SEQ ID NO: 84 - DNA, 21 bp, synthetic construct
  source 1..21: mol_type = other DNA
cgccatggagattggagatgt

SEQ ID NO: 85 - DNA, 20 bp, synthetic construct
  source 1..20: mol_type = other DNA
catgaagatcagggaatgga

SEQ ID NO: 86 - DNA, 23 bp, synthetic construct
  source 1..23: mol_type = other DNA
taacatgaagatcagagaatgga

SEQ ID NO: 87 - DNA, 1001 bp, Zea mays
  source 1..1001: mol_type = genomic DNA
  variation 501: note = m = a or c
acaaaccacgattacacatacaattggatctaatctaacaaaccaagatggttgcttgtacgcttgcacctaataactgcttgcatattagtcattgccattctgctcacaggtccgtacatatgtattttttattcatatgaaccatttaagttcggtttttcagttaaaccggaaaacaaaccgaattatacgattaaccgaaggttcggtttttccttttttaaatagaatttcggttgatgttcttcagaaactaaactttgaaaaaaccgaaagaaccgaaccgaaaaacttcggttaaccgactacctggtcctagtaaccaaacaccaccaattatttcgtcaaacagcttctgagccttctgtagattctcatcaacagcccactcagagatcaggatggtttaggttttcgccgagacatcaaactgaaatggcgactgcctaaagaatgcctcgctctaccaggccattatgcgataacatgaagatcagmgaatggagatcagcgaccctcggcaggaaagtaggaaaccgaacatctccaatctccatggcggagaaagctcggatggcatcgtcgcgggatatcggatagcagagagcgcagaagcggaagagccgcgccccgcaagcgaaccggcgagaatgagaagcgagcccagaaggtgtgtgaacggtgaaccccggcagcgcggcggcgaagaggaagaaccggagggagggcacgggaaggttgcggcagccgccgctgcggacgtgaggcgtggcgcggaggtcgtgccccggcccgcgttagttgctgagcacacggcagagcgcgccgacgagccgcggatcggggcccggggccggatcgggccgcggatccagctgtccggcgggcgaggcggagtagaagccgcacaggaggagagaacgcgggcttcgcggcacggccgcagcgcggagcctggcggcagatttcatctcgcgccgatgtgggcgcagggggtt

SEQ ID NO: 88 - DNA, 19 bp, synthetic construct
  source 1..19: mol_type = other DNA
gtcgcgatgcttcatgctt

SEQ ID NO: 89 - DNA, 22 bp, synthetic construct
  source 1..22: mol_type = other DNA
tcagtgcaaagtgcaggtaatg

SEQ ID NO: 90 - DNA, 22 bp, synthetic construct
  source 1..22: mol_type = other DNA
cctgttaggtttacagtagaat

SEQ ID NO: 91 - DNA, 23 bp, synthetic construct
  source 1..23: mol_type = other DNA
cctgttaggtttacaatagaatt

SEQ ID NO: 92 - DNA, 1001 bp, Zea mays
  source 1..1001: mol_type = genomic DNA
  variation 141: note = n = a or c or g or t/u
  variation 183: note = n = a or c or g or t/u
  variation 216: note = n = a or c or g or t/u
  variation 267: note = n = a or c or g or t/u
  variation 306: note = n = a or c or g or t/u
  variation 318: note = n = a or c or g or t/u
  variation 501: note = r = a or g
  variation 647: note = n = a or c or g or t/u
  variation 842: note = n = a or c or g or t/u
gcagaagaatcaggcttcacaatgccctcgagaatgtcgatgtagtggtctggggggtttacgcggtcaggaacatggattccaagagctgtgaagtactcctcgaccgtctttactgggccattgtagaccatcaacccnccttttgcgaggagtatcaaatcgtcgaacatgttgtacagngtgtagctcggttgatgaacaacagcacagacnttcacgccttggagcgcctcatgccgaagggctctgaggagaagttgtgangaggagctatccaggccagatgttggttcatccaggatnagaagcgacggntccatcaccatctcgatgcccacattgacacgcttcctctgcccgccagaaatcccacgcttttcaactgtgccaaccaatgaatttctgattccctgaagatccagagaatctatcactctctccactaggagcaccttgtcgcgatgcttcatgcttgctgatagcctgttaggtttacartagaattgttaagattgctgctagtgaggtaactaactacagggttgaatttaatttaaaatcattacctgcactttgcactgaaccagaggttttcctccaccgttaagtttccatgaacaatgtcatcttgtggcacaaatccnatgacctttttgtaggaacggatattaagattctttccattaacaagaacagaacctgtcatcttgtagccgttcaccttcccagtgacggcattgagaaatgtggtcttgccagcccctgatgggcccatgacagcagttacacgccccggccggagttcaccattgatagacctcagcagcttcttcttcccnagcatcagtgtcaggtctctgaactcaaccttgagcaaaggcctagttatctccttctgctggtccttcaccatggcgatcaccccagtaaaagttagattcttgttgtcctgtcgctgctggaatttctccttctcaatctcgccatacgcatacttg

SEQ ID NO: 93 - DNA, 19 bp, synthetic construct
  source 1..19: mol_type = other DNA
catcaggacagggcaggaa

SEQ ID NO: 94 - DNA, 18 bp, synthetic construct
  source 1..18: mol_type = other DNA
gtccgcgccaagttcaag

SEQ ID NO: 95 - DNA, 17 bp, synthetic construct
  source 1..17: mol_type = other DNA
ttgggaagcagcacatc

SEQ ID NO: 96 - DNA, 18 bp, synthetic construct
  source 1..18: mol_type = other DNA
ttgggaagcaacacatca

SEQ ID NO: 97 - DNA, 1001 bp, Zea mays
  source 1..1001: mol_type = genomic DNA
  variation 501: note = r = a or g
attgatgaggaccatgtcattttccttgactagagtgaagaacagtgaaaagtttttaacttgcgttcttcactaaagttaattactccctcagccagctataataagctcaccgaattcaggccagtatcaacatgagcacaacaaaccacaatagccaaactaccatgattctcagctgaatagggactatttgctgctacagactcaatgtgcctcctaatcctaacaaacaaccgtaaccacaaactatgatgcgtttacaacctaaagatctagcctctacagcaatgtcgagtgcaaactaagcagaattcgttgccatctcatttgatagcaaacaacaaattcaagatcaaccagccaaacaaatagagaataacagacgtctgtggatcatacctaaccttagatgctgctcgggtacatgaacactctgaccttgcgcccctgcactcaaatgtaaaaaggcatcaggacagggcaggaattgggaagcarcacatcacatagcttagggggttcggcacagaccatggactcaggcgggaggttggacttgaacttggcgcggacgacgcccgagttgccgtgcgggcgggtgaccttgccccagatgcacctgtagcgggtctcgctgctcttggtcttagccttgtagatgtacgccatgcgcttgccagcgtaccacgcgacgtcctccttggtgttcaccccctcgatctgaatgagcgacgtggtctcgtactggttcgacttggacctgcatagccaccaccgcaaccactgtcagcggaatcgaaccgagaaaggccagatgtatgtagacggagaagaagcggcggcgcggatctcacctcttgtatccgaggatggtgccccggacgtagagcctgacgcgctgtcccgtgcgtcccttcaccatggtggccgcctgcttccttccacttctgcttccgccgccggagacgagagagagctcgtgcaggtttatatgtagc

SEQ ID NO: 98 - DNA, 22 bp, synthetic construct
  source 1..22: mol_type = other DNA
aaggcaaaaggagggaactgat

SEQ ID NO: 99 - DNA, 21 bp, synthetic construct
  source 1..21: mol_type = other DNA
tccttgttccgtcttttgcag

SEQ ID NO: 100 - DNA, 15 bp, synthetic construct
  source 1..15: mol_type = other DNA
tacctcggcgacgcc

SEQ ID NO: 101 - DNA, 21 bp, synthetic construct
  source 1..21: mol_type = other DNA
tccttgttccgtcttttgcag

SEQ ID NO: 102 - DNA, 22 bp, synthetic construct
  source 1..22: mol_type = other DNA
aaggcaaaaggagggaactgat

SEQ ID NO: 103 - DNA, 12 bp, synthetic construct
  source 1..12: mol_type = other DNA
ccggcgtctcgc

SEQ ID NO: 104 - DNA, 13 bp, synthetic construct
  source 1..13: mol_type = other DNA
cgcgctcacgcac

SEQ ID NO: 105 - DNA, 26 bp, synthetic construct
  source 1..26: mol_type = other DNA
caggttcttgagtctcggaatattca

SEQ ID NO: 106 - DNA, 25 bp, synthetic construct
  source 1..25: mol_type = other DNA
gatcaccgtcaacttcagagtatgt

SEQ ID NO: 107 - DNA, 16 bp, synthetic construct
  source 1..16: mol_type = other DNA
tttgatgcagctcaca

SEQ ID NO: 108 - DNA, 18 bp, synthetic construct
  source 1..18: mol_type = other DNA
tctttgatgctgctcaca

SEQ ID NO: 109 - DNA, 22 bp, synthetic construct
  source 1..22: mol_type = other DNA
cagagatggtggtggtggatgt

SEQ ID NO: 110 - DNA, 24 bp, synthetic construct
  source 1..24: mol_type = other DNA
caaagggtatcactggaacaggac

SEQ ID NO: 111 - DNA, 14 bp, synthetic construct
  source 1..14: mol_type = other DNA
ttgatggcacggga

SEQ ID NO: 112 - DNA, 17 bp, synthetic construct
  source 1..17: mol_type = other DNA
tttgatggcatgggaag

SEQ ID NO: 113 - DNA, 20 bp, synthetic construct
  source 1..20: mol_type = other DNA
gccctcatgcacctcatacc

SEQ ID NO: 114 - DNA, 24 bp, synthetic construct
  source 1..24: mol_type = other DNA
gacgacgaacataagaggaggatt

SEQ ID NO: 115 - DNA, 15 bp, synthetic construct
  source 1..15: mol_type = other DNA
acgaccttcgtaacg

SEQ ID NO: 116 - DNA, 16 bp, synthetic construct
  source 1..16: mol_type = other DNA
acgacctttgtaacgt

SEQ ID NO: 117 - DNA, 20 bp, synthetic construct
  source 1..20: mol_type = other DNA
gccctcatgcacctcatacc

SEQ ID NO: 118 - DNA, 20 bp, synthetic construct
  source 1..20: mol_type = other DNA
caccgtcgccctatcagtct

SEQ ID NO: 119 - DNA, 17 bp, synthetic construct
  source 1..17: mol_type = other DNA
aaggtcgtaccaaatcc

SEQ ID NO: 120 - DNA, 14 bp, synthetic construct
  source 1..14: mol_type = other DNA
cgcggaccaaatcc

SEQ ID NO: 121 - DNA, 21 bp, synthetic construct
  source 1..21: mol_type = other DNA
tcaagaggggggagaactacc

SEQ ID NO: 122 - DNA, 19 bp, synthetic construct
  source 1..19: mol_type = other DNA
gtttccccgcaaaaagctc

SEQ ID NO: 123 - DNA, 16 bp, synthetic construct
  source 1..16: mol_type = other DNA
cgctcttttaaaataa

SEQ ID NO: 124 - DNA, 16 bp, synthetic construct
  source 1..16: mol_type = other DNA
cgctctttaaaaataa

SEQ ID NO: 125 - DNA, 20 bp, synthetic construct
  source 1..20: mol_type = other DNA
acaagatagggccgttcaca

SEQ ID NO: 126 - DNA, 16 bp, synthetic construct
  source 1..16: mol_type = other DNA
gaaatgcgcccgacca

SEQ ID NO: 127 - DNA, 20 bp, synthetic construct
  source 1..20: mol_type = other DNA
tgaagaatggattttagctt

SEQ ID NO: 128 - DNA, 19 bp, synthetic construct
  source 1..19: mol_type = other DNA
aagaatggattttcgcttt

SEQ ID NO: 129 - DNA, 22 bp, synthetic construct
  source 1..22: mol_type = other DNA
caagtcactgcttccgtccatt

SEQ ID NO: 130 - DNA, 21 bp, synthetic construct
  source 1..21: mol_type = other DNA
cgaggatcgacgctttgttca

SEQ ID NO: 131 - DNA, 15 bp, synthetic construct
  source 1..15: mol_type = other DNA
taccatcgacttttc

SEQ ID NO: 132 - DNA, 15 bp, synthetic construct
  source 1..15: mol_type = other DNA
accatcgatttttca

SEQ ID NO: 133 - DNA, 19 bp, synthetic construct
  source 1..19: mol_type = other DNA
ccctgttccgaggccattt

SEQ ID NO: 134 - DNA, 20 bp, synthetic construct
  source 1..20: mol_type = other DNA
cgtgagctttcgtctgcgat

SEQ ID NO: 135 - DNA, 17 bp, synthetic construct
  source 1..17: mol_type = other DNA
tcgatcgggtatgctct

SEQ ID NO: 136 - DNA, 14 bp, synthetic construct
  source 1..14: mol_type = other DNA
cgggtgtgctctcc

SEQ ID NO: 137 - DNA, 18 bp, synthetic construct
  source 1..18: mol_type = other DNA
ctgcacgccgccagatta

SEQ ID NO: 138 - DNA, 22 bp, synthetic construct
  source 1..22: mol_type = other DNA
cagctgacggcaacaattagta

SEQ ID NO: 139 - DNA, 18 bp, synthetic construct
  source 1..18: mol_type = other DNA
cgaccgtaaccatgtaac

SEQ ID NO: 140 - DNA, 17 bp, synthetic construct
  source 1..17: mol_type = other DNA
accgtaaccctgtaaca

SEQ ID NO: 141 - DNA, 25 bp, synthetic construct
  source 1..25: mol_type = other DNA
atggtgccaattcgtaatttaagtt

SEQ ID NO: 142 - DNA, 20 bp, synthetic construct
  source 1..20: mol_type = other DNA
acccctctggttgcctctct

SEQ ID NO: 143 - DNA, 16 bp, synthetic construct
  source 1..16: mol_type = other DNA
aataccctccagtttc

SEQ ID NO: 144 - DNA, 20 bp, synthetic construct
  source 1..20: mol_type = other DNA
tatgaagaagaataccatcc

SEQ ID NO: 145 - DNA, 23 bp, synthetic construct
  source 1..23: mol_type = other DNA
ggctaatatggtgccaattcgta

SEQ ID NO: 146 - DNA, 23 bp, synthetic construct
  source 1..23: mol_type = other DNA
ggagtatacagacccctctggtt

SEQ ID NO: 147 - DNA, 20 bp, synthetic construct
  source 1..20: mol_type = other DNA
ttgaaactggagggtattct

SEQ ID NO: 148 - DNA, 20 bp, synthetic construct
  source 1..20: mol_type = other DNA
cttgaaactggatggtattc

SEQ ID NO: 149 - DNA, 21 bp, synthetic construct
  source 1..21: mol_type = other DNA
gtattcgcacctactctgccg

SEQ ID NO: 150 - DNA, 23 bp, synthetic construct
  source 1..23: mol_type = other DNA
aaagcaaaaataccattgcaacc

SEQ ID NO: 151 - DNA, 19 bp, synthetic construct
  source 1..19: mol_type = other DNA
cgtaaattttgtttgatgc

SEQ ID NO: 152 - DNA, 21 bp, synthetic construct
  source 1..21: mol_type = other DNA
atcgtaaattttttttgatgc

SEQ ID NO: 153 - DNA, 25 bp, synthetic construct
  source 1..25: mol_type = other DNA
ggctttatagtcctcagaaaggtga

SEQ ID NO: 154 - DNA, 21 bp, synthetic construct
  source 1..21: mol_type = other DNA
tcgtagccacgtgctctaatc

SEQ ID NO: 155 - DNA, 18 bp, synthetic construct
  source 1..18: mol_type = other DNA
cgggaagagatcctgtgg

SEQ ID NO: 156 - DNA, 14 bp, synthetic construct
  source 1..14: mol_type = other DNA
cccgggacctgtgg

SEQ ID NO: 157 - DNA, 22 bp, synthetic construct
  source 1..22: mol_type = other DNA
cagtgcgcctcatacagttgta

SEQ ID NO: 158 - DNA, 18 bp, synthetic construct
  source 1..18: mol_type = other DNA
accaagccggcaaaggat

SEQ ID NO: 159 - DNA, 16 bp, synthetic construct
  source 1..16: mol_type = other DNA
catgtgcctaggagag

SEQ ID NO: 160 - DNA, 16 bp, synthetic construct
  source 1..16: mol_type = other DNA
catgtgcctcggagag

SEQ ID NO: 161 - DNA, 22 bp, synthetic construct
  source 1..22: mol_type = other DNA
caagtcactgcttccgtccatt

SEQ ID NO: 162 - DNA, 22 bp, synthetic construct
  source 1..22: mol_type = other DNA
ggatcgacgctttgttcacctg

SEQ ID NO: 163 - DNA, 18 bp, synthetic construct
  source 1..18: mol_type = other DNA
cacgacgaaaggattaaa

SEQ ID NO: 164 - DNA, 18 bp, synthetic construct
  source 1..18: mol_type = other DNA
cacgacgaaaggatttaa

SEQ ID NO: 165 - DNA, 25 bp, synthetic construct
  source 1..25: mol_type = other DNA
ggcttatagcttagaggcacttgaa

SEQ ID NO: 166 - DNA, 18 bp, synthetic construct
  source 1..18: mol_type = other DNA
cctctgcaccgccatcaa

SEQ ID NO: 167 - DNA, 14 bp, synthetic construct
  source 1..14: mol_type = other DNA
tcagcctcatcctc

SEQ ID NO: 168 - DNA, 15 bp, synthetic construct
  source 1..15: mol_type = other DNA
ctcagccttatcctc

SEQ ID NO: 169 - DNA, 19 bp, synthetic construct
  source 1..19: mol_type = other DNA
gtggccattgcatgtaagc

SEQ ID NO: 170 - DNA, 25 bp, synthetic construct
  source 1..25: mol_type = other DNA
tgatgtggtagtttctggattgaac

SEQ ID NO: 171 - DNA, 21 bp, synthetic construct
  source 1..21: mol_type = other DNA
atatgtactttccctcacaag

SEQ ID NO: 172 - DNA, 18 bp, synthetic construct
  source 1..18: mol_type = other DNA
tgtactttccgtcacaag

SEQ ID NO: 173 - DNA, 21 bp, synthetic construct
  source 1..21: mol_type = other DNA
gcagacgtgaagaggatagca

SEQ ID NO: 174 - DNA, 21 bp, synthetic construct
  source 1..21: mol_type = other DNA
tccctcggaacacaacaatgg

SEQ ID NO: 175 - DNA, 14 bp, synthetic construct
  source 1..14: mol_type = other DNA
tgtcgctcgaagcc

SEQ ID NO: 176 - DNA, 15 bp, synthetic construct
  source 1..15: mol_type = other DNA
ctgtcgctggaagcc

SEQ ID NO: 177 - DNA, 170 bp, Zea mays
  source 1..170: mol_type = genomic DNA
  variation 21: note = n = a or c or g or t/u
ctctccgaccctggtgctaancacgttccttgttccgtcttttgcaggtggtgcgagagcgcgtgcgtgcggggatcagttccctccttttgccttttttgtgggctgctcttacttgcttgcaagctgtttgatggaatgcaggaaagggctgctgggaccgggggaag